(19) United States
(12) Patent Application Publication
(10) Pub. No.: US 2025/0098702 A1
(43) Pub. Date: Mar. 27, 2025

(54) Preparation Of Pulse Protein Products ("YP870")

(71) Applicant: Burcon Nutrascience (MB) Corp., Winnipeg (CA)

(72) Inventors: Martin Schweizer, Winnipeg (CA); Randy Willardsen, Roseville, CA (US)

(21) Appl. No.: 18/832,653

(22) PCT Filed: Jan. 24, 2023
(86) PCT No.: PCT/CA2023/050082

§ 371 Date: Jul. 24, 2024

Related U.S. Application Data
(60) Provisional application No. 63/302,306, filed on Jan. 24, 2022.

(51) Int. Cl. A23J 1/14; A23J 3/14

(52) U.S. Cl. CPC A23J 1/14; A23J 3/14

(57) ABSTRACT

A process for preparing a pulse protein product from a pulse protein source, the pulse protein product having a protein content of greater than 60 wt % (N×6.25) d.b, is provided. A pulse protein product is also provided as are food and beverages, pet food, animal feed, industrial product, cosmetic product or personal care product comprising the pulse protein product.

### RELATED APPLICATION

This application claims priority to U.S. Provisional Patent Application 63/302,306 filed Jan. 24, 2022, herein incorporated by reference in its entirety.

### FIELD OF THE INVENTION

The present invention relates to novel and inventive pulse protein products and to novel and inventive methods of preparing pulse protein products.

### SUMMARY OF INVENTION

The present invention relates to novel and inventive pulse products, processes for the preparation thereof and products comprising them.

In one embodiment, the present invention provides for a process for preparing a pulse protein product from a pulse protein source, the pulse protein product having a protein content of greater than 60 wt % (N×6.25) d.b., the process comprising:

extracting pulse protein from a pulse protein source to cause solubilization of the pulse protein from the pulse protein source to produce an aqueous phase and a residual pulse protein source,

separating the aqueous phase from the residual pulse protein source to produce an aqueous pulse protein solution and a separated residual pulse protein source,

optionally adjusting the pH of the aqueous pulse protein solution to a higher value not exceeding about pH 8.0;

optionally concentrating the aqueous pulse protein solution to provide a concentrated pulse protein solution having a protein concentration of about 5 to about 30 wt %, preferably about 5 to about 20 wt %, more preferably about 10 to about 20 wt %;

optionally diafiltering the concentrated pulse protein solution using water as the diafiltration solution OR diafiltering the aqueous pulse protein solution prior to concentration or diafiltering the partially concentrated aqueous pulse protein solution;

optionally drying the pulse protein solution or optionally concentrated and/or optionally diafiltered pulse protein solution to provide a pulse protein product;

wherein steps e) and f) may optionally be carried out simultaneously.

In a further embodiment of the process or process outlined above, the process further comprises:

treating the aqueous pulse protein solution with an anti-foamer, such as any suitable food-grade, non-silicone based anti-foamer, to reduce the volume of foam formed upon further processing and wherein the anti-foamer is optionally added during the extraction step a).

In a further embodiment of the process or process outlined above, the quantity of anti-foamer employed is generally greater than about 0.0003% w/v.

In a further embodiment of the process or process outlined above, the process further comprises:

a. treating the aqueous pulse protein solution with an adsorbent, such as granulated activated carbon, to remove colour and/or odour compounds.

In a further embodiment of the process or process outlined above, separation comprises centrifugation and/or filtration optionally with a decanter centrifuge and a disc stack centrifuge.

In a further embodiment of the process or process outlined above, the process further comprises the step of defatting the separated aqueous pulse protein solution to at least partially remove fat from the separated aqueous pulse protein solution, optionally by centrifugation and/or filtration.

In a further embodiment of the process or process outlined above, solubilization from the pulse protein source is effected using water having different levels of purity, such as tap water or reverse osmosis (RO) purified water.

In a further embodiment of the process or process outlined above, a pH of the extraction may be the natural pH of the combination of the water and the pulse protein source, or the pH of the extraction may be adjusted up to any value between the natural pH and about 8.0, or the pH may be adjusted within the range of about 6.8 to about 8.0, or preferably the pH may be adjusted to about 6.8 to about 7.5.

In a further embodiment of the process or process outlined above, food grade sodium hydroxide, potassium hydroxide or any other conventional food grade alkali or combinations thereof are added to the water to adjust the pH of the extraction.

In a further embodiment of the process or process outlined above, solubilization of the protein is effected at a temperature of from about 1° to about 100° C., preferably about 15° to about 65° C., more preferably about 20° C. to about 35° C. or about 50° to about 60° C., preferably accompanied by agitation.

In a further embodiment of the process or process outlined above, the solubilization time is about 1 to about 60 minutes, preferably about 10 to about 30 minutes.

In a further embodiment of the process or process outlined above, the concentration of pulse protein source in the water during the extraction step is about 5 to about 20% w/v.

In a further embodiment of the process or process outlined above, the aqueous phase resulting from the extraction step generally has a protein concentration of about 0.5 to about 5 wt %, preferably about 1 to about 5 wt %.

In a further embodiment of the process or process outlined above, the water of extraction may contain an antioxidant, such as ascorbic acid, optionally in an amount of from about 0.01 to about 1 wt % of the solution, preferably about 0.05 to about 0.15 wt %.

In a further embodiment of the process or process outlined above, separation step b) is conducted at the same temperature as the extraction step or at any temperature within the range of about 1° to about 100° C., preferably about 15° to about 65° C., more preferably about 50° to about 60° C.

In a further embodiment of the process or process outlined above, the concentration step d) is effected by selective membrane technique, such as ultrafiltration or diafiltration, using membranes, such as hollow-fibre membranes or spiral-wound membranes, with a suitable molecular weight cut-off, such as about 1,000 to about 1,000,000 daltons, preferably about 1,000 to about 100,000 daltons, more preferably about 10,000 to about 100,000 daltons,

In a further embodiment of the process or process outlined above, the diafiltration step e) is effected using water as diafiltration solution without any pH adjustment or the water is adjusted with any suitable food grade alkali to raise the pH of the diafiltration solution to a value between the pH of the water and the pH of the optionally concentrated pulse protein solution.

In a further embodiment of the process or process outlined above, diafiltration is effected using from about 1 to about 40 volumes of diafiltration solution, preferably about 2 to about 25 volumes of diafiltration solution, more preferably about 2 to about 5 volumes of diafiltration solution.

In a further embodiment of the process or process outlined above, diafiltration is effected using the same membrane as for the concentration step d) or the diafiltration step e) is effected using a separate membrane with a different molecular weight cut-off, such as a membrane having a molecular weight cut-off in the range of about 1,000 to about 1,000,000 daltons, preferably about 1,000 to about 100,000 daltons, more preferably about 10,000 to about 100,000 daltons.

In a further embodiment of the process or process outlined above, a further concentration step is applied after the diafiltration step

In a further embodiment of the process or process outlined above, the concentration step d) and/or the diafiltration step e) are effected in such a manner that the pulse protein product subsequently recovered contains at least about 60 wt %, at least about 65 wt %, at least about 70 wt %, at least about 75 wt %, at least about 80 wt %, at least about 85 wt %, or greater than about 90 wt % protein (N×6.25) d.b.

In a further embodiment of the process or process outlined above, the diafiltration water comprises an antioxidant, such as ascorbic acid, optionally in an amount of from about 0.01 to about 1 wt %, preferably about 0.05 to about 0.15 wt %.

In a further embodiment of the process or process outlined above, the optional concentration step d) and the optional diafiltration step e) are effected at generally about 2° to about 65° C., preferably about 50° to about 60° C.

In a further embodiment of the process or process outlined above, the concentrated and/or diafiltered protein solution is subject to a defatting step or a further defatting step to that defined above.

In a further embodiment of the process or process outlined above, the concentrated and/or diafiltered protein solution are treated with an adsorbent, such as granulated activated carbon, to remove colour and/or odour compounds.

In a further embodiment of the process or process outlined above, the concentration and/or diafiltration steps are operated in a manner favourable for removal of trypsin inhibitors in the permeate, optionally by using a membrane of larger pore size, such as 30,000 to 1,000,000 Da, operating the membrane at elevated temperatures, such as about 30° to about 65° C., preferably about 50° to about 60° C. and employing greater volumes of diafiltration medium, such as 10 to 40 volumes.

In a further embodiment of the process or process outlined above, the aqueous pulse protein solution is exposed to reducing agents that at least partially disrupt or rearrange the disulfide bonds of the inhibitors, such as cysteine or N-acetylcysteine.

In a further embodiment of the process or process outlined above, the optionally concentrated and optionally diafiltered aqueous pulse protein solution is pasteurized prior to optional drying or further processing and wherein pasteurization optionally comprises heating the optionally concentrated and optionally diafiltered pulse protein solution to a temperature of about 55° to about 75° C. for about 15 seconds to about 60 minutes or about 55° to about 85° C. for about 10 seconds to about 60 minutes, preferably about 60° to about 70° C. for about 10 minutes to about 60 minutes or about 70° to about 85° C. for about 10 seconds to about 60 seconds, and optionally the pasteurized pulse protein solution is cooled, such as to a temperature of about 20° to about 35° C.

In a further embodiment of the process or process outlined above, the optionally concentrated and optionally diafiltered aqueous pulse protein solution is jet cooked prior to optional drying to a temperature of about 110° C. to about 150° C. for about 10 seconds to about 1 minute, preferably to about 135° C. to 145° C. for about 40 to 50 seconds.

In a further embodiment of the process or process outlined above, when the separation step b) is done in more than one step (e.g. employing decanter centrifuge then disc stack centrifuge such as defined above), different temperatures may be employed for each step of the separation process, preferably about 20 to about 60° C., more preferably about 20 to about 35° C. or about 50 to about 60° C. for the initial separation step (decanter centrifuge) and about 50 to about 85° C., more preferably about 50 to about 80° C., most preferably about 50 to about 60° C. for the second separation step (disc stack centrifuge).

In a further embodiment of the process or process outlined above, the temperature of the protein solution is raised after the first separation step and held for at least 1 minute, preferably at least 5 minutes, before the second separation step.

In a further embodiment of the process or process outlined above, the temperature of the protein solution is raised after the first separation step to about 65° to about 85° C., preferably about 65° to about 80° C.

In a further embodiment of the process or process outlined above, the temperature of the protein solution is raised after the first separation step and subsequently lowered before the second separation step.

In a further embodiment of the process or process outlined above, the temperature of the protein solution is lowered to about 50° to about 60° C. before the second separation step.

In a further embodiment of the process or process outlined above, the optionally concentrated, optionally diafiltered and optionally pasteurized pulse protein solution is subject to drying step f) by any conventional means such as spray drying or freeze drying to provide a pulse protein product.

In a further embodiment of the process or process outlined above, the process further comprises:

bi) optionally further processing the separated residual pulse protein source obtained in step b) such as to recover residual protein; or
bii) optionally re-extracting the separated residual pulse protein source obtained in step b) with fresh water to recover residual protein and separating the re-extraction protein solution from the residual pulse protein source and optionally combining the re-extraction protein solution with the aqueous pulse protein solution for further processing; or
biii) using the separated residual pulse protein source obtained in step b) in food products, pet foods, animal feed and in industrial, cosmetic and personal care products; or
biv) drying the separated residual pulse protein source obtained in step b) for disposal or for future use in food products, pet foods, animal feed and in industrial, cosmetic and personal care products.

In a further embodiment of the process or process outlined above, extraction step a) is carried out using a counter-current extraction procedure.

In a further embodiment of the process or process outlined above, the finer solids are captured separately from the bulk of the separated residual pulse protein source in step b), optionally by the disc stack centrifuge, are optionally diluted with water, optionally RO water, then optionally dried to form a pulse protein product having a protein content of at least about 50 wt % (N×6.25) d.b., preferably at least about 55, 60, 65, 70 and 75 wt % (N×6.25) d.b., more preferably at least about 80 wt % (N×6.25) d.b.

In a further embodiment of the process or process outlined above, the pH of the optionally diluted finer solids is raised to a value between the natural pH of the solids and a pH of about 8.0, by any conventional means such as by the addition of sodium hydroxide, potassium hydroxide or any other conventional food grade alkali or combination thereof prior to optional drying to form a pulse protein product having a protein content of at least about 50 wt % (N×6.25) d.b., preferably at least about 55, 60, 65, 70 and 75 wt % (N×6.25) d.b., more preferably at least about 80 wt % (N×6.25) d.b.

In a further embodiment of the process or process outlined above, the finer solids captured separately from the bulk of the separated residual pulse protein source in step b), optionally by the disc stack centrifuge, are washed in order to remove contaminants and improve the purity and flavour of the product, optionally by suspending the solids in between about 1 and about 20 volumes, preferably about 1 to about 10 volumes, more preferably between about 1 and 5 volumes of wash solution such as water, preferably RO water, and optionally the washing step is conducted at any conventional temperature such as about 1° to about 75° C., preferably about 15° to about 65° C., more preferably about 50° to about 60° C., and optionally for any conventional length of time, preferably 15 minutes or less, then the washed finer solids are separated from the used wash solution by any convenient means such as centrifugation using a disc stack centrifuge.

In a further embodiment of the process or process outlined above, the used wash solution separated from the finer solids is optionally added to the aqueous pulse protein solution arising from the separation step b) for further processing.

In a further embodiment of the process or process outlined above, the washed finer solids are optionally diluted with water then optionally dried by any conventional means such as spray drying or freeze drying to provide a pulse protein product having a protein content of at least about 50 wt % (N×6.25) d.b., preferably at least about 55, 60, 65, 70, 75, 80 and 85 wt % (N×6.25) d.b., more preferably about 90 wt % (N×6.25) d.b.

In a further embodiment of the process or process outlined above, the pH of the optionally diluted washed finer solids is adjusted to a value between the natural pH of the mixture of finer solids and water and about 8.0, by any conventional means such as by the addition of sodium hydroxide, potassium hydroxide or any other conventional food grade alkali and combinations thereof, prior to optional drying.

In a further embodiment of the process or process outlined above, the finer solids are pH adjusted during the washing step by adjusting the mixture of finer solids and wash solution to a pH between the natural pH of the mixture and about 8.0 using food grade alkali, then separating the washed solids from the used wash solution by centrifugation and optionally diluting and optionally drying the washed solids.

In a further embodiment of the process or process outlined above, the used wash solution is added to the aqueous pulse protein solution arising from the separation step b) for further processing.

In a further embodiment of the process or process outlined above, the process further comprises pasteurizing the optionally diluted and optionally pH adjusted finer solids or optionally diluted and optionally pH adjusted washed finer solids prior to the optional drying step, wherein pasteurization optionally comprises heating to a temperature of about 55° to about 75° C. for about 15 seconds to about 60 minutes or about 55° to about 85° C. for about 10 seconds to about 60 minutes, preferably about 60° to about 70° C. for about 10 minutes to about 60 minutes or about 70° to about 85° C. for about 10 seconds to about 60 seconds.

In a further embodiment of the process or process outlined above, the pasteurized optionally diluted and optionally pH adjusted finer solids or optionally diluted and optionally pH adjusted washed finer solids is cooled, such as to a temperature of about 20° to about 35° C.

In a further embodiment of the process or process outlined above, the optionally diluted and optionally pH adjusted finer solids or optionally diluted and optionally pH adjusted washed finer solids are jet cooked prior to optional drying to a temperature of about 110° C. to about 150° C. for a time of about 10 seconds to about 1 minute, preferably to about 135° C. to 145° C. for about 40 to 50 seconds.

In a further embodiment of the process or process outlined above, the extraction is carried out in a continuous operation or a batch operation.

In a further embodiment, the present invention provides for a pulse protein product with a sodium content of less than about 0.40% d.b. or preferably less than about 0.30% d.b.

In a further embodiment of the product or products outlined above, the sodium content is less than about 0.12% d.b.

In a further embodiment, the present invention provides for a pulse protein product with viscosity for a 20 wt % protein solution of less than about 10,000 mPa·s when measured at a shear rate of 100 (1/s).

In a further embodiment of the product or products outlined above, the viscosity is less than about 6,389 mPa·s when measured at a shear rate of 100 (1/s).

In a further embodiment, the present invention provides for a pulse protein product having a solubility of less than about 60% when measured at pH 4, less than about 25% when measured at pH 5.5 and less than about 80% when measured at pH 7.

In a further embodiment of the product or products outlined above, the product has a solubility of about 9.1 to about 54.6% when measured at pH 4, about 5.3 to about 20.4% when measured at pH 5.5 and about 20.3 to about 76.7% when measured at pH 7.

In a further embodiment of the product or products outlined above, the product has a solubility of about 22.5 to about 54.6% when measured at pH 4, about 5.8 to about 20.4% when measured at pH 5.5 and about 20.3 to about 68.1% when measured at pH 7 and wherein the pulse protein product is optionally yellow pea.

In a further embodiment of the product or products outlined above, the product has a solubility of about 6.6 to about 20.2% when measured at pH 4, about 0.7 to about 10.1% when measured at pH 5.5 and about 7.0 to about 36.2% when measured at pH 7.

In a further embodiment, the present invention provides for a pulse protein product wherein the dry colour L* value is about 77.70 to about 97.99, the a* value is about −0.44 to about 2.03 and the b* value is about 14.34 to about 26.32.

In a further embodiment of the product or products outlined above, the dry colour L* value is about 77.70 to about 82.83, the a* value is about 0.58 to about 2.03 and the b* value is about 16.40 to about 26.32 and wherein the pulse protein product is optionally yellow pea.

In a further embodiment, the present invention provides for a pulse protein product or a residual sunflower protein product having an attribute from one or more of the following tables:

a solubility as defined in or captured by Table 3,

a dry colour as defined in or captured by Table 4;

a water binding capacity as defined or captured by Table 5;

an oil binding capacity as defined in or captured by Table 6;

a phytic acid content as defined in or captured by Table 7;

a sodium content as defined in or captured by Table 8;

an amino acid profile comprising one or more amino acids as defined or captured by Table 9;

a viscosity for a 20 wt % protein solution as defined in or captured by Table 10.

In a further embodiment of the product or products outlined above, the product is derived from any pulse as defined herein.

In a further embodiment of the product or products outlined above, the product is derived from yellow peas.

In a further embodiment of the product or products outlined above, or in an embodiment of the process or processes outlined above, the pulse protein product is derived from a pulse source selected from lentils, chickpeas, dry peas, dry beans, lupines, bambara beans, broad or fava beans, dry pigeon peas, vetches, dried cowpeas, winged beans, sword beans and yellow peas and wherein the pulse source is optionally dehulled.

In a further embodiment of the product or products outlined above, the product may be prepared by a process or processes as outlined above or herein.

In a further embodiment, the present invention provides for a food or beverage comprising a pulse protein product as outlined above or herein.

In a further embodiment of the food or beverage or beverages as outlined above, the food or beverage is:

a dairy alternative;

a meat alternative;

a seafood alternative;

a grain product;

a snack or sweet;

a fats and oils product;

a condiment or sauce; or

a nutritional product.

In a further embodiment of the food or beverage or beverages as outlined above, the dairy alternative is:

a milk alternative beverage;

a frozen dessert;

a cheese alternative; or

a yogurt alternative.

In a further embodiment of the food or beverage or beverages as outlined above, the meat alternative is:

a beef alternative;

a pork alternative; or

a poultry alternative.

In a further embodiment of the food or beverage or beverages as outlined above, the seafood alternative is:

a tuna alternative;

a salmon alternative; or

a shrimp alternative.

In a further embodiment of the food or beverage or beverages as outlined above, the grain product is:

a pasta;

a bread; or

a breakfast cereal.

In a further embodiment of the food or beverage or beverages as outlined above, the snack or sweet is:

a cookie;

a cracker;

a bar product;

a cake;

a candy; or

a chocolate.

In a further embodiment of the food or beverage or beverages as outlined above, the fats and oils is:

a margarine; or

a dressing.

In a further embodiment of the food or beverage or beverages as outlined above, the condiment or sauce is:

a tomato based sauce;

a non tomato based sauce;

a dip; or

a gravy.

In a further embodiment of the food or beverage or beverages as outlined above, the nutritional product is:

a nutritional drink; or

a nutritional powder.

In a further embodiment of the food or beverage or beverages as outlined above, the beverage is:

a sports drink;

an energy drink; or

a smoothie.

In a further embodiment, the present invention provides for a pet food, animal feed, industrial product, cosmetic product or personal care product comprising a pulse protein product as outlined above or herein.

### DETAILED DESCRIPTION

The initial step of the process of providing the pulse protein products of the present invention involves solubilizing pulse protein from a pulse protein source. The pulses to which the present invention may be applied include, but are not limited to, lentils, chickpeas, dry peas, dry beans, lupines, bambara beans, broad or fava beans, dry pigeon peas, vetches, dried cowpeas, winged beans, sword beans. Some examples of pulses are described in “Pulses Nutritious Seeds For A Sustainable Future”, Food and Agriculture Organization of the United Nations, 2016, herein incorporated by reference. A further example of pulses are yellow peas. The pulse protein source may be pulses or any pulse product or by-product derived from the processing of pulses. For example, the pulse protein source may be a flour prepared by grinding an optionally dehulled pulse. As another example, the pulse protein source may be a protein-rich pulse fraction formed by dehulling and grinding a pulse and then air classifying the dehulled and ground material into starch-rich and protein-rich fractions. The pulse protein product recovered from the pulse protein source may be the protein naturally occurring in pulses or the proteinaceous material may be a protein modified by genetic manipulation but possessing characteristic hydrophobic and polar properties of the natural protein.

The pulse protein products of the present invention may be prepared from pulse protein source by either a batch process or a continuous process or a semi-continuous process. Protein solubilisation from the pulse protein source material is effected using water. The water used may be tap water or water having different levels of purity. In an embodiment of the present invention, reverse osmosis (RO) purified water is preferred.

The pH of the extraction may be the natural pH of the combination of the water and the pulse protein source, or the pH of the extraction may be adjusted up to any value between the natural pH and about 8.0, preferably the pH is adjusted within the range of about 6.8 to about 8.0, more preferably the pH is adjusted to about 6.8 to about 7.5. Food grade sodium hydroxide, potassium hydroxide or any other conventional food grade alkali or combinations thereof may be added to the water to adjust the pH of the extraction as required. The food grade alkali is preferably added in aqueous solution form.

The solubilization of the protein is effected at a temperature of from about 1° to about 100° C., preferably about 15° to about 65° C., more preferably about 20° C. to about 35° C. or about 50° to about 60° C., preferably accompanied by agitation to decrease the solubilization time, which is usually about 1 to about 60 minutes, preferably about 10 to about 30 minutes. The temperature of the extraction should be such that the viscosity of the slurry of pulse protein source in water does not significantly impair mixing or pumpability. It is preferred to effect the solubilization to extract substantially as much protein from the pulse protein source as is practicable, so as to provide an overall high product yield. The pH values of the extraction and subsequent steps refer to values typically measured at room temperature (about 21 to about 24° C.). For absence of doubt, when, for example, the extraction is conducted at an elevated temperature, the pH of the extraction mixture is such that a sample of extraction mixture cooled to room temperature has a pH reading in the specified range.

Extraction of the protein from the pulse protein source, when conducted in a continuous operation, is carried out in any manner consistent with effecting a continuous extraction of protein from the pulse protein source. In one embodiment, the pulse protein source is continuously mixed with the water and the mixture is conveyed through a pipe or conduit having a length and at a flow rate for a residence time sufficient to effect the desired extraction in accordance with the parameters described herein.

The concentration of pulse protein source in the water during the solubilization step may vary widely. Typical concentration values are about 5 to about 20% w/v.

It will be appreciated that reference to solubilizing encompasses both complete and partial solubilization of the protein from the pulse protein source.

The protein extraction step has the additional effect of solubilizing fats which may be present in the pulse protein source, which then results in the fats being present in the aqueous phase.

The protein solution resulting from the extraction step generally has a protein concentration of about 0.5 to about 5 wt %, preferably about 1 to about 5 wt %.

The water of extraction may contain an antioxidant. The antioxidant may be any conventional antioxidant, such as ascorbic acid. The quantity of antioxidant employed may vary from about 0.01 to about 1 wt % of the solution, preferably about 0.05 to about 0.15 wt %. The antioxidant serves to inhibit oxidation of any phenolics in the protein solution.

The aqueous phase resulting from the extraction step then may be separated from the residual pulse protein source, in any conventional manner, such as by centrifugation and/or filtration. This separation step may be conducted with for example a decanter centrifuge to remove the bulk of the residual solids, with finer residual solids remaining in the protein solution. Preferably the aqueous phase resulting from the extraction step is separated from the bulk of the residual pulse protein source using a decanter centrifuge and the resulting centrate further clarified using a disc stack centrifuge to remove finer solids. These finer solids may be combined with the bulk of the residual pulse protein source for further processing or may be further processed on their own to provide a product of the invention as described below in greater detail. The separation step may be conducted at the same temperature as the extraction step or at any temperature within the range of about 1° to about 100° C., preferably about 15° to about 65° C., more preferably about 50° to about 60° C. When the separation is done in more than one step (e.g. employing decanter centrifuge then disc stack centrifuge), different temperatures within the abovementioned range may be employed for each step of the separation process, preferably about 20 to about 60° C., more preferably about 20 to about 35° C. or about 50 to about 60° C. for the initial separation step (decanter centrifuge) and preferably about 50 to about 85° C., more preferably about 50 to about 80° C., most preferably about 50 to about 60° C. for the second separation step (disc stack centrifuge). When the temperature of the protein solution arising from the first separation step is raised prior to the second separation step, an optional hold time may be applied at the elevated temperature, such as for at least about 1 to about 5 minutes. If desired, the protein solution arising from the first separation step may be raised in temperature, optionally held for at least about 1 to about 5 minutes, and then cooled to a temperature in the abovementioned range prior to the second separation step (disc stack centrifuge). The temperatures employed in the separation step(s) should be such that the viscosity of the slurry of pulse protein source in water does not significantly impede the separation step. The separated residual pulse protein source material (with or without the finer solids) may be used as-is in food products, pet foods, animal feed and in industrial, cosmetic and personal care products, dried for disposal or for future use in food products, pet foods, animal feed and in industrial, cosmetic and personal care products or further processed, such as to recover starch and/or residual protein. Residual protein may be recovered by re-extracting the separated residual pulse protein source with fresh water and the protein solution yielded upon clarification combined with the initial protein solution for further processing as described below. A counter-current extraction procedure may also be utilized. The separated residual pulse protein source may alternatively be processed by any other conventional procedure to recover residual protein.

When the separation is done in one step, such as to leave the finer solids in the protein solution, the separated protein solution may be heat treated, such as to about 65 to about 85° C., preferably about 65 to about 80° C. with an optional hold time of at least about 1 minute or at least about 5 minutes. Such a heat treatment at this step of the procedure (or between separation steps as described above) is believed to improve the organoleptic properties of the final product. The heat treated protein solution may be cooled for further processing.

It will be appreciated that reference herein to a separation step and to separation of the aqueous phase and residual pulse protein source is intended to refer to both complete separation as well as to at least partial separation. It is to be understood that trace or minor amounts of residual components may be found in the aqueous phase, for example but not limited to: residual protein source, finer solids, and/or fat/oil.

The aqueous pulse protein solution may be treated with an anti-foamer, such as any suitable food-grade, non-silicone based anti-foamer, to reduce the volume of foam formed upon further processing. The quantity of anti-foamer employed is generally greater than about 0.0003% w/v. Alternatively, the anti-foamer in the quantity described may be added in the extraction steps.

The separated aqueous pulse protein solution may be subject to a defatting operation, if desired or required. Defatting of the separated aqueous pulse protein solution may be achieved by any conventional procedure such as centrifugation and/or filtration.

The aqueous pulse protein solution may be treated with an adsorbent, such as granulated activated carbon, to remove colour and/or odour compounds. Such adsorbent treatment may be carried out under any conventional conditions, generally at the ambient temperature of the separated aqueous protein solution.

The aqueous pulse protein solution may be adjusted in pH to a higher value not exceeding about pH 8.0. The increase in pH is believed to improve the flux rate of subsequent membrane processing.

If of adequate purity, the aqueous pulse protein solution may be directly dried to produce a pulse protein product having a protein content of greater than 60 wt % (N×6.25) d.b. To provide a pulse protein product having a decreased impurities content, such as a pulse protein isolate, the aqueous pulse protein solution may be processed as described below prior to drying. Further processing as described below is also believed to have a beneficial effect on the flavour of the product.

The aqueous pulse protein solution may be concentrated to provide a concentrated pulse protein solution having a protein concentration of about 5 to about 30 wt %, preferably about 5 to about 20 wt %, more preferably about 10 to about 20 wt %. It will be appreciated that concentrations of less than about 5 wt % may be considered as partially concentrated.

The concentration step may be effected in any conventional manner consistent with batch or continuous operation, such as by employing any conventional selective membrane technique, such as ultrafiltration or diafiltration, using membranes, such as hollow-fibre membranes or spiral-wound membranes, with a suitable molecular weight cut-off, such as about 1,000 to about 1,000,000 daltons, preferably about 1,000 to about 100,000 daltons, more preferably about 10,000 to about 100,000 daltons having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass therethrough while preventing higher molecular weight species from so doing. The low molecular weight species include low molecular weight materials extracted from the source material, such as carbohydrates, pigments, low molecular weight proteins and anti-nutritional factors, such as trypsin inhibitors, which are themselves low molecular weight proteins. The molecular weight cut-off of the membrane is usually chosen to ensure retention of a significant proportion of the protein in the solution, while permitting contaminants to pass through having regard to the different membrane materials and configurations.

The concentrated pulse protein solution then may be subjected to a diafiltration step using water as the diafiltration solution. The water may be used as the diafiltration solution without any pH adjustment or any suitable food grade alkali may be added to the water to raise the pH of the diafiltration solution to a value between the pH of the water and the pH of the concentrated pulse protein solution. Preferably, the pH of the diafiltration solution is equal to that of the protein solution being diafiltered. The diafiltration may also be done in stages using water at different pH values, such as initial volumes of diafiltration using water adjusted to the pH of the protein solution followed by additional volumes of diafiltration water without pH adjusting agent. Such diafiltration may be effected using from about 0.5 to about 40 volumes of diafiltration solution, preferably about 1 to about 40 volumes of diafiltration solution, more preferably about 2 to about 25 volumes of diafiltration solution, most preferably about 2 to about 5 volumes of diafiltration solution. In the diafiltration operation, further quantities of contaminants are removed from the aqueous pulse protein solution by passage through the membrane with the permeate. This purifies the aqueous protein solution and may also reduce its viscosity. The diafiltration operation may be effected until no significant further quantities of contaminants or visible colour are present in the permeate or until the retentate has been sufficiently purified so as to provide a pulse protein isolate with a protein content of at least about 90 wt % (N×6.25) d.b. Such diafiltration may be effected using the same membrane as for the concentration step. However, if desired, the diafiltration step may be effected using a separate membrane with a different molecular weight cut-off, such as a membrane having a molecular weight cut-off in the range of about 1,000 to about 1,000,000 daltons, preferably about 1,000 to about 100,000 daltons, more preferably about 10,000 to about 100,000 daltons having regard to different membrane materials and configuration. If desired, the diafiltered protein solution may be further concentrated.

Alternatively, the diafiltration step may be applied to the aqueous protein solution prior to concentration or to partially concentrated aqueous protein solution. Diafiltration may also be applied at multiple points during the concentration process. When diafiltration is applied prior to concentration or to the partially concentrated solution, the resulting diafiltered solution may then be additionally concentrated. Diafiltering multiple times as the protein solution is concentrated may allow a higher final, fully concentrated protein concentration to be achieved. This reduces the volume of material to be dried. Concentration and diafiltration may also be conducted simultaneously.

The concentration step and the diafiltration step may be effected herein in such a manner that the pulse protein product subsequently recovered contains less than about 90 wt % protein (N×6.25) d.b., such as at least about 60 wt % protein (N×6.25) d.b. By partially concentrating and/or partially diafiltering the aqueous pulse protein solution, it is possible to only partially remove contaminants. This protein solution may then be dried to provide a pulse protein product with lower levels of purity.

An antioxidant may be present in the diafiltration water during at least part of the diafiltration step. The antioxidant may be any conventional antioxidant, such as ascorbic acid. The quantity of antioxidant employed in the diafiltration water depends on the materials employed and may vary from about 0.01 to about 1 wt %, preferably about 0.05 to about 0.15 wt %. The antioxidant serves to inhibit the oxidation of any phenolics present in the pulse protein solution.

The optional concentration step and the optional diafiltration step may be effected at any conventional temperature, generally about 2° to about 65° C., preferably about 50° to about 60° C., and for the period of time to effect the desired degree of concentration and diafiltration. The temperature and other conditions used to some degree depend upon the membrane equipment used to effect the membrane processing, the desired protein concentration of the solution and the efficiency of the removal of contaminants to the permeate.

Pulses contain anti-nutritional trypsin inhibitors. The level of trypsin inhibitor activity in the final pulse protein product can be controlled by the manipulation of various process variables.

The concentration and/or diafiltration steps may be operated in a manner favourable for removal of trypsin inhibitors in the permeate along with the other contaminants. Removal of the trypsin inhibitors is promoted by using a membrane of larger pore size, such as 30,000 to 1,000,000 Da, operating the membrane at elevated temperatures, such as about 30° to about 65° C., preferably about 50° to about 60° C. and employing greater volumes of diafiltration medium, such as 10 to 40 volumes.

Further, a reduction in trypsin inhibitor activity may be achieved by exposing pulse materials to reducing agents that disrupt or rearrange the disulfide bonds of the inhibitors. Suitable reducing agents include, but are not limited to cysteine and N-acetylcysteine.

The addition of such reducing agents may be effected at various stages of the overall process. The reducing agent may be added with the pulse protein source material in the extraction step, may be added to the aqueous pulse protein solution following removal of residual pulse protein source material, may be added to the diafiltered retentate before drying or may be dry blended with the dried pulse protein product. The addition of the reducing agent may be combined with the membrane processing steps, as described above.

If it is desired to retain active trypsin inhibitors in the protein solution, this can be achieved by not utilizing reducing agents, utilizing a concentration and diafiltration membrane with a smaller pore size, operating the membrane at lower temperatures and employing fewer volumes of diafiltration medium.

The concentrated and/or diafiltered protein solution may be subject to a further defatting operation, if required. Defatting of the concentrated and/or diafiltered protein solution may be achieved by any conventional procedure.

The concentrated and/or diafiltered acidified aqueous protein solution may be treated with an adsorbent, such as granulated activated carbon, to remove colour and/or odour compounds. Such adsorbent treatment may be carried out under any conventional conditions, generally at the ambient temperature of the protein solution.

The optionally concentrated and optionally diafiltered aqueous pulse protein solution may be pasteurized prior to drying or further processing. Such pasteurization may be effected under any conventional pasteurization conditions. Generally, the optionally concentrated and optionally diafiltered pulse protein solution is heated to a temperature of about 55° to about 75° C. for about 15 seconds to about 60 minutes or about 55° to about 85° C. for about 10 seconds to about 60 minutes, preferably about 60° to about 70° C. for about 10 minutes to about 60 minutes or about 70° to about 85° C. for about 10 seconds to about 60 seconds. The pasteurized pulse protein solution then may be cooled, such as to a temperature of about 20° to about 35° C.

The optionally concentrated and optionally diafiltered aqueous pulse protein solution may be jet cooked prior to drying in order to modify the functional properties of the protein product. In such jet cooking, the protein solution may be heated to a temperature of about 110 to about 150° C. for a time of about 10 seconds to about 1 minute. Preferably the solution is heated to about 135° C. to 145° C. for about 40 to 50 seconds.

The optionally concentrated, optionally diafiltered optionally pasteurized and optionally jet cooked pulse protein solution then may be dried by any conventional means such as spray drying or freeze drying to provide a pulse protein product having a protein content greater than about 60 wt % d.b. Preferably the pulse protein product has a protein content greater than about 65, 70, 75, 80 and 85 wt % d.b. Most preferably, the pulse protein product is an isolate with a protein content in excess of about 90 wt % protein (N×6.25) d.b.

The pulse protein product prepared from the solution of soluble protein has organoleptic and functional properties making it suitable for use in various food and beverage products. Based on its water binding capacity, the pulse protein product may have utility in meat alternatives (for example beef alternatives, pork alternatives, poultry alternatives and the like) and bakery applications (for example breads, cookies, cakes and the like). Other food uses for the pulse protein product include but are not limited to dairy alternatives (for example beverages, frozen desserts, cheese and yogurt alternatives and the like), seafood alternatives (for example tuna alternatives, salmon alternatives, shrimp alternatives and the like), grain products other than those indicated as bakery applications (for example pastas, breakfast cereals and the like), snacks and sweets other than those indicated as bakery applications (for example crackers, bar products, candies, chocolates and the like), beverages (for example sports drinks, energy drinks, smoothies and the like), fats and oils products (for example margarines, dressings and the like), condiments and sauces (for example tomato based or other sauces, dips, gravies and the like) and nutritional products (for example drinks, powders and the like). The pulse protein product may be formulated into a food or beverage product to provide protein fortification. The pulse protein product may be formulated into a food or beverage product to replace other protein ingredients (including as an extender in meat or dairy products) or to replace non-protein functional ingredients. The pulse protein product prepared from the solution of soluble protein may also be used in pet foods, animal feed and in industrial, cosmetic and personal care products.

In accordance with another aspect of the present invention, the finer solids captured by the disc stack centrifuge in the separation step may be further processed to provide a pulse protein product. The finer solids may be optionally diluted with RO water then optionally dried to form a pulse protein product having a protein content of at least about 50 wt % (N×6.25) d.b., preferably at least about 55, 60, 65, 70, and 75 wt % (N×6.25) d.b., more preferably at least about 80 wt % (N×6.25) d.b. Alternatively, the pH of the optionally diluted finer solids may be raised to a value between the natural pH of the solids and a pH of about 8.0, by any conventional means such as by the addition of sodium hydroxide, potassium hydroxide or any other conventional food grade alkali or combination thereof prior to optional drying to form a pulse protein product having a protein content of at least about 50 wt % (N×6.25) d.b., preferably at least about 55, 60, 65, 70 and 75 wt % (N×6.25) d.b., more preferably at least about 80 wt % (N×6.25) d.b. The food grade alkali is preferably added in aqueous solution form.

Preferably, the finer solids are washed in order to remove contaminants and improve the purity and flavour of the product. The finer solids may be washed by suspending the solids in between about 1 and about 20 volumes, preferably about 1 to about 10 volumes, more preferably between about 1 and 5 volumes of water, preferably RO water. The washing step may be conducted at any conventional temperature such as about 1° to about 75° C., preferably about 15° to about 65° C., more preferably about 50° to about 60° C. The washing step is conducted for any conventional length of time, preferably 15 minutes or less. The finer solids may then be separated from the used wash solution by any conventional means such as by centrifugation using a disc stack centrifuge. The used wash solution may be added to the protein solution arising from the initial separation step for further processing as described above. The washed finer solids may be optionally diluted with water then optionally dried by any conventional means such as spray drying or freeze drying to provide a pulse protein product having a protein content of at least about 50 wt % (N×6.25) d.b., preferably at least about 55, 60, 65, 70, 75, 80 and 85 wt % (N×6.25) d.b., more preferably at least about 90 wt % (N×6.25) d.b. Alternatively, the pH of the optionally diluted washed finer solids may be adjusted to a value between the natural pH of the mixture of finer solids and water and about 8.0, by any conventional means such as by the addition of sodium hydroxide, potassium hydroxide or any other conventional food grade alkali and combinations thereof, prior to optional drying. As a further alternative, the finer solids may be pH adjusted during the washing step by adjusting the mixture of finer solids and wash water to a pH between the natural pH of the mixture and about 8.0 using food grade alkali, then collecting the solids by centrifugation and optionally diluting and optionally drying the solids. The food grade alkali is preferably added in aqueous solution form.

A pasteurization step may be employed on the optionally diluted and optionally pH adjusted finer solids or optionally diluted and optionally pH adjusted washed finer solids prior to the optional drying step. Such pasteurization may be effected under any conventional pasteurization conditions. Generally, the optionally diluted and optionally pH adjusted finer solids or optionally diluted and optionally pH adjusted washed finer solids are heated to a temperature of about 55° to about 75° C. for about 15 seconds to about 60 minutes or about 55° to about 85° C. for about 10 seconds to about 60 minutes, preferably about 60° to about 70° C. for about 10 minutes to about 60 minutes or about 70° to about 85° C. for about 10 seconds to about 60 seconds. The pasteurized optionally diluted and optionally pH adjusted finer solids or optionally diluted and optionally pH adjusted washed finer solids then may be cooled, such as to a temperature of about 20° to about 35° C.

The optionally diluted and optionally pH adjusted finer solids or optionally diluted and optionally pH adjusted washed finer solids may be jet cooked prior to drying in order to modify the functional properties of the protein product. In such jet cooking, the protein material may be heated to a temperature of about 110 to about 150° C. for a time of about 10 seconds to about 1 minute. Preferably the material is heated to about 135° C. to 145° C. for about 40 to 50 seconds.

The product derived from the finer solids may be lower in purity compared to the product derived from the protein solution. However, the pulse protein product prepared from the finer solids has organoleptic and functional properties making it suitable for use in various food and beverage products as described above for the pulse protein product prepared from the aqueous pulse protein solution. The pulse protein product may be formulated into a food or beverage product to provide protein fortification. The pulse protein product may be formulated into a food or beverage product to replace other protein ingredients or to replace non-protein functional ingredients. The pulse protein product prepared from the finer solids may also be used in pet foods, animal feed and in industrial, cosmetic and personal care products.

### EXAMPLES

### Example 1

‘a’ kg of yellow split pea flour was combined with ‘b’ L of RO water having a temperature of ‘c’ ° C. and the mixture was stirred for 10 minutes. A ‘d’ kg portion of the suspended residual solids were removed by centrifugation using a decanter centrifuge to provide a protein solution having a protein content of ‘c’ wt %. The protein solution was then heated to about ‘f’ ° C. and centrifuged with a disc stack centrifuge to provide ‘g’ L of protein solution and ‘h’ kg of finer residual solids. The pH of the protein solution was adjusted from ‘i’ to ‘j’ with ‘k’ kg of 25% NaOH.

The pH adjusted protein solution had a protein content of ‘l’ wt % and a dry matter content of ‘m’ wt %. As such, the dry basis protein content of the material was calculated to be ‘n’ wt % (N×6.25) d.b. The pH adjusted solution was reduced in volume from ‘o’ to ‘p’ L by concentration on a polyethersulfone membrane having a molecular weight cutoff (MWCO) of 100,000 daltons operated at a temperature of about ‘q’ ° C. The concentrated protein solution with a protein content of ‘r’ wt % was then diafiltered on the same membrane with ‘s’ L of RO water at a temperature of about ‘t’ ° C. The diafiltered protein solution was further concentrated to a protein content of ‘u’ wt %. This protein solution was pasteurized at about 72° C. for 16 seconds. The pasteurized material was spray dried to yield a product having a protein content of ‘v’ % (N×6.25) d.b. This product was termed ‘w’ YP870N.

The finer residual solids had a protein content of ‘x’ wt %. A ‘y’ kg aliquot of finer residual solids was freeze dried to yield a product having an as-is protein content of ‘z’ % (N×6.25). A ‘aa’ kg aliquot of finer residual solids was pH adjusted to ‘ab’ with ‘ac’ kg of 25% NaOH solution and then pasteurized at ‘ad’ ° C. for ‘ae’ minute. ‘af’ kg of pasteurized material was spray dried to yield a product having a protein content of ‘ag’ % (N×6.25) d.b. This product was termed ‘w’ YP870PN.

Note that all cited pH values were from measurements conducted with the sample at room temperature unless otherwise noted.

TABLE 1
Process parameters for preparation of pulse protein products

| w | MTYP61-F29-21A | MTYP62-H26-21A |
|---|---|---|
| a | 48.1 | 96 |
| b | 300 | 600 |
| c | 19.8 | 22.3 |
| d | 81.84 | 164.68 |
| e | 2.83 | 2.49 |
| f | 50 | 52 |
| g | 230 | 500 |
| h | 17.46 | 19.38 |
| i | 6.45 | 6.40 |
| j | 7.44 | 7.43 |
| k | 0.58 | 0.54 |
| l | 2.45 | 2.13 |
| m | 3.52 | not determined |
| n | 69.60 | not determined |
| o | 220 | 500 |
| p | 65 | 120 |
| q | 39 | 48 |
| r | 7.02 | 8.34 |
| s | 325 | 600 |
| t | 33 | 46 |
| u | 10.50 | 12.56 |
| v | 92.10 | 91.49 |
| x | 6.86 | 8.70 |
| y | 0.5095 | not applicable |
| z | 68.47 | not applicable |
| aa | not applicable | 19.38 |
| ab | not applicable | 6.93 |
| ac | not applicable | 0.02 |
| ad | not applicable | 72 |
| ae | not applicable | 1 |
| af | not applicable | 16.64 |
| ag | not applicable | 76.02 |

### Example 2

96 kg of yellow split pea flour was combined with 600 L of RO water having a temperature of about 23° C. and 1.02 kg of 25% NaOH solution and mixed for 10 minutes. 162.9 kg of suspended residual solids were removed by centrifugation using a decanter centrifuge to provide a protein solution having a protein content of 2.75 wt % and a pH of 7.34. The protein solution was then heated to about 52° C. and centrifuged with a disc stack centrifuge to provide 460 L of protein solution having a pH of 7.39 and 16 kg of finer residual solids.

The protein solution, having a protein content of 2.50 wt % was reduced in volume from 460 to 140 L by concentration on a polyethersulfone membrane having a MWCO of 100,000 daltons operated at a temperature of about 51° C. The concentrated protein solution, having a protein content of 7.59 wt %, was then diafiltered on the same membrane with 700 L of RO water at about 51° C., then further concentrated to a protein content of 14.06 wt %. This protein solution was diluted with 15 L of RO water and pasteurized at about 72° C. for 16 seconds. The pasteurized material was diluted with 7 L of RO water and spray dried to yield a product having a protein content of 90.26% (N×6.25) d.b. This product was termed MTYP62-102-21A YP870N.

The finer residual solids, having a protein content of 6.31 wt %, was pasteurized at about 72° C. for 1 minute and then spray dried to yield a product having a protein content of 74.72% (N×6.25) d.b. This product was termed MTYP62-102-21A YP870PN.

Note that all cited pH values were from measurements conducted with the sample at room temperature unless otherwise noted.

### Example 3

96 kg of yellow split pea flour was combined with 600 L of RO water having a temperature of ‘a’ ° C. and ‘b’ kg of 25% NaOH solution and mixed for 10 minutes. An ‘c’ kg portion of suspended residual solids were removed by centrifugation using a decanter centrifuge to provide a protein solution having a protein content of ‘d’ wt % and a pH of ‘e’. The protein solution was then heated to about ‘f’ ° C. and centrifuged with a disc stack centrifuge to provide ‘g’ L of protein solution having a pH of ‘h’ and ‘i’ kg of finer residual solids.

The protein solution was pH adjusted with ‘j’ kg of 25% NaOH solution to ‘k’ and had a protein content of ‘l’ wt %. The pH adjusted solution was reduced in volume from ‘m’ to ‘n’ L by concentration on a polyethersulfone membrane having a MWCO of 100,000 daltons operated at a temperature of about ‘o’ ° C. The concentrated protein solution had a protein content of ‘p’ wt %. ‘q’ kg of concentrated protein solution was discarded and the remainder was then diafiltered on the same membrane with ‘r’ L of RO water at about ‘s’ ° C., then further concentrated to a protein content of ‘t’ wt %. The membrane system was flushed with RO water to recover more protein. ‘u’ L of system flush was added to the concentrated protein solution. This protein solution was pasteurized at about 72° C. for 16 seconds. The pasteurized material was spray dried to yield a product having a protein content of ‘v’ % (N×6.25) d.b. This product was termed ‘w’ YP870N.

The finer residual solids, having a protein content of ‘x’ wt %, was pasteurized at about 72° C. for 1 minute and then spray dried to yield a product having a protein content of ‘y’ % (N×6.25) d.b. This product was termed ‘w’ YP870PN.

Note that all cited pH values were from measurements conducted with the sample at room temperature unless otherwise noted.

TABLE 2
Process parameters for preparation of pulse protein products

| w | MTYP62-I08-21A | MTYP62-I09-21A |
|---|---|---|
| a | 23 | 22.7 |
| b | 0.44 | 0.42 |
| c | 163.1 | 164.7 |
| d | 2.66 | 2.70 |
| e | 6.84 | 6.82 |
| f | 50 | 52 |
| g | not recorded | 530 |
| h | 6.78 | 6.79 |
| i | 13.28 | 16.2 |
| j | not recorded | 0.42 |
| k | 7.28 | 7.35 |
| l | 2.32 | 2.37 |
| m | not recorded | 530 |
| n | 170 | 145 |
| o | 48 | 49 |
| p | 7.44 | 8.18 |
| q | 0 | 80 |
| r | 850 | 140 |
| s | 51 | 54 |
| t | 16.66 | 14.30 |
| u | 14.5 | 13.16 |
| v | 92.13 | 88.66 |
| x | 7.84 | 7.70 |
| y | 80.98 | 74.70 |

### Example 4

48 kg of yellow split pea flour was combined with 300 L of RO water having a temperature of 54.7° C. and 0.18 kg of 25% NaOH solution and mixed for 10 minutes. 81.25 kg of suspended residual solids were removed by centrifugation using a decanter centrifuge to provide a protein solution having a protein content of 2.99 wt % and a pH of 6.70. The protein solution was then heated to 66.1° C., held for at least 5 minutes and then centrifuged with a disc stack centrifuge to provide 245 L of protein solution having a protein content of 2.56 wt % protein as well as 12.30 kg of finer residual solids. The pH of the protein solution was then adjusted from 6.67 to 7.69 by the addition of 0.24 kg of 25% NaOH solution.

The pH adjusted protein solution was reduced in volume from 245 to 75 L by concentration on a polyethersulfone membrane having a MWCO of 100,000 daltons operated at a temperature of about 53° C. The concentrated protein solution with a protein content of 7.87 wt % was then diafiltered on the same membrane with 375 L of RO water with the diafiltration operation conducted at about 52° C. then further concentrated to a protein content of 13.41 wt %. This protein solution (39.56 kg) was pasteurized at about 72° C. for 60 seconds. 37.18 kg of pasteurized solution was spray dried to yield a product having a protein content of 90.78% (N×6.25) d.b. This product was termed MTYP63-129-21A YP870N.

The 12.30 kg of finer residual solids with a protein content of 7.53 wt % was combined with 49.2 kg of RO water having a temperature of 50° C. The mixture was centrifuged with a disc stack centrifuge and 11.84 kg of washed finer residual solids having a protein content of 4.27 wt % and 50 L of used wash solution having a protein content of 0.64 wt % protein was collected. The washed finer residual solids were pasteurized at about 72° C. then spray dried to yield a product having a protein content of 81.41% (N×6.25) d.b. This product was termed MTYP63-129-21A YP870PN.

Note that all cited pH values were from measurements conducted with the sample at room temperature unless otherwise noted.

Example 5

48 kg of yellow split pea flour was combined with 300 L of RO water having a temperature of 51.5° C. and 0.22 kg of 25% NaOH solution and mixed for about 10 minutes. An 81.95 kg portion of suspended residual solids were removed by centrifugation using a decanter centrifuge to provide a protein solution having a protein content of 3.25 wt % and a pH of 6.83. The protein solution was then heated to 64.9° C., held for at least 5 minutes and then centrifuged with a disc stack centrifuge to provide 240 L of protein solution having a protein content of 2.99 wt % protein as well as 10.08 kg of finer residual solids.

10 kg of finer residual solids with a protein content of 6.57 wt % was combined with 40 L of RO water having a temperature of 52.3° C. The mixture was centrifuged with a disc stack centrifuge and 7.60 kg of washed finer residual solids and 38 L of used wash solution having a protein content of 0.64 wt % protein was collected.

The protein solution was combined with the used wash solution and the pH of the mixture adjusted to 7.22 by the addition of 0.14 kg of 25% NaOH solution. The pH adjusted protein solution, having a protein content of 2.77 wt %, was reduced in volume from 270 to 90 L by concentration on a polyethersulfone membrane having a MWCO of 100,000 daltons operated at a temperature of about 53° C. The concentrated protein solution with a protein content of 7.32 wt % was then diafiltered on the same membrane with 450 L of RO water with the diafiltration operation conducted at about 52° C. then further concentrated to a protein content of 14.11 wt %. This protein solution was pasteurized at about 72° C. for 16 seconds. 43.3 kg of pasteurized protein solution was spray dried to yield a product having a protein content of 91.80% (N×6.25) d.b. This product was termed MTYP64-J07-21A YP870N.

7.6 kg of the washed residual finer solids, having a protein content of 3.74 wt % and a solids content of 4.39 wt % (85.19% protein (N×6.25) d.b.) was pasteurized at about 72° C. for 16 seconds and then spray dried to yield a product termed MTYP64-J07-21A YP870PN.

Note that all cited pH values were from measurements conducted with the sample at room temperature unless otherwise noted.

### Example 6

48 kg of yellow split pea flour was combined with 300 L of RO water having a temperature of 51.3° C. and 25% NaOH solution to adjust the pH of the mixture. Another 48 kg of yellow split pea flour was combined with 300 L of RO water having a temperature of 48.1° C. and 25% NaOH solution to adjust the pH of the mixture. In total 0.46 kg of 25% NaOH solution was used. The mixtures were stirred for 10 minutes. A 163.28 kg portion of suspended residual solids were removed by centrifugation using a decanter centrifuge to provide a protein solution having a protein content of 2.88 wt % and a pH of 6.75. The protein solution was then heated to 66.8° C., held for at least 5 minutes then centrifuged with a disc stack centrifuge to provide 490 L of protein solution having a protein content of 2.61 wt % as well as 20.24 kg of finer residual solids.

20.24 kg of finer residual solids with a protein content of 7.87 wt % was combined with 80 L of RO water having a temperature of 50° C. The mixture was centrifuged with a disc stack centrifuge and 11.76 kg of washed finer residual solids and 100 L of used wash solution having a protein content of 0.82 wt % was collected.

The protein solution was combined with the used wash solution and the pH of the mixture adjusted to 7.45 by the addition of 0.42 kg of 25% NaOH solution. The pH adjusted solution, having a protein content of 2.36 wt %, was reduced in volume from 590 L to 172 L by concentration on a polyethersulfone membrane having a MWCO of 100,000 daltons operated at a temperature of about 51° C. The concentrated protein solution with a protein content of 7.01 wt % was then diafiltered on the same membrane with 850 L of RO water with the diafiltration operation conducted at about 50° C. then further concentrated to a protein content of 13.00 wt %. This protein solution was pasteurized at about 72° C. for 16 seconds. 33.60 kg of pasteurized solution was spray dried to yield a product having a protein content of 92.21% (N×6.25) d.b. This product was termed MTYP63-J13-21A YP870N.

11.76 kg of the washed finer residual solids, having a protein content of 5.53 wt %, was pasteurized at 72° C. for 60 seconds then discarded.

Note that all cited pH values were from measurements conducted with the sample at room temperature unless otherwise noted.

### Example 7

48 kg of yellow split pea flour was combined with 300 L of RO water having a temperature of 52.5° C. and 25% KOH solution to adjust the pH of the mixture. Another 48 kg of yellow split pea flour was combined with 300 L of RO water having a temperature of 51.9° C. and 25% KOH solution to adjust the pH of the mixture. In total 0.98 kg of 25% KOH solution was used. The mixtures were stirred for 10 minutes. A 169.14 kg portion of suspended solids were removed by centrifugation using a decanter centrifuge to provide a protein solution having a protein content of 3.29 wt % and a pH of 6.98. The protein solution was then heated to 66.5° C., held for at least 5 minutes and then centrifuged with a disc stack centrifuge to provide 520 L of protein solution at 3.00 wt % protein and 20.54 kg of finer residual solids.

20.54 kg of finer residual solids with a protein content of 7.96 wt % was combined with 82.16 kg of RO water having a temperature of 50° C. The mixture was centrifuged with a disc stack centrifuge and 11.80 kg of washed finer residual solids and 100 L of used wash solution having a protein content of 0.82 wt % was collected.

The protein solution was combined with the used wash solution and the pH adjusted with a 25% NaOH solution to 7.51 resulting in a protein solution containing 2.62 wt % protein. The pH adjusted solution was reduced in volume from 620 to 203 L by concentration on a polyethersulfone membrane having a MWCO of 100,000 daltons operated at a temperature of about 51° C. The concentrated protein solution with a protein content of 6.65 wt % was then diafiltered on the same membrane with 1000 L of RO water with the diafiltration operation conducted at about 49° C. then further concentrated to a protein content of 15.73 wt %. 90 kg of this protein solution was pasteurized at about 72° C. for 16 seconds. A 60 kg aliquot of pasteurized protein solution was then spray dried to yield a product having a protein content of 92.48% (N×6.25) d.b. This product was termed MTYP64-J14-21A YP870N.

11.80 kg of washed residual finer solids, having a protein content of 5.67 wt % and a solids content of 6.88 (82.41% protein (N×6.25) d.b.) was pasteurized at about 73° C. for 60 seconds. An 8.0 kg aliquot of the pasteurized solids was spray dried to yield a product termed MTYP64-J14-21A YP870PN.

A 30 kg aliquot of the pasteurized protein solution and a 4 kg aliquot of the pasteurized solids were combined and the resulting material had a protein solution of 13.21 wt %. The material was spray dried to yield a product having a protein content of 92.14% (N×6.25) d.b. This product is termed MTYP64-J14-21A YP870N+PN.

Note that all cited pH values were from measurements conducted with the sample at room temperature unless otherwise noted.

### Example 8

48 kg of yellow split pea flour was combined with 300 L of RO water having a temperature of 55.8° C. and 25% NaOH solution to adjust the pH of the mixture. Another 48 kg of yellow split pea flour was combined with 300 L of RO water having a temperature of 56.1° C. and 25% NaOH solution to adjust the pH of the mixture. In total 0.38 kg of 25% NaOH solution was used. The mixtures were stirred for 10 minutes. A 171.38 kg portion of suspended residual solids were removed by centrifugation using a decanter centrifuge to provide a protein solution having a protein content of 3.39 wt % and a pH of 6.89. The protein solution was then heated to 65.9° C., held for at least 5 minutes and then centrifuged with a disc stack centrifuge to provide 490 L of protein solution at 3.14 wt % protein as well as 19.80 kg of finer residual solids.

A 7.0 kg portion of the finer residual solids from the disc stack centrifuge with a protein content of 8.33 wt % was pasteurized at about 72° C. for 60 seconds and then spray dried to yield a product having a protein content of 72.30% (N×6.25) d.b. This product was termed MTYP64-K08-21A YP870PN-01.

Another 12.7 kg of finer residual solids was combined with 50.8 L of RO water having a temperature of 53.9° C. The mixture was centrifuged with a disc stack centrifuge and 7.76 kg of washed finer residual solids and 90 L of used wash solution at 0.57 wt % protein was collected. 7.76 kg of the washed finer residual solids from the disc stack centrifuge having a protein content of 4.85 wt % was pasteurized at about 72° C. for 60 seconds and then spray dried to yield a product having a protein content of 79.48% (N×6.25) d.b. This product was termed MTYP64-K08-21A YP870PN-02

The protein solution was combined with the used wash solution and pH adjusted to 7.55 resulting in a protein solution containing 2.66 wt % protein. The pH adjusted solution was reduced in volume from 580 to 185 L by concentration on a polyethersulfone membrane having a MWCO of 100,000 daltons operated at a temperature of about 52° C. The concentrated protein solution with a protein content of 7.40 wt % was then diafiltered on the same membrane with 925 L of RO water with the diafiltration operation conducted at about 53° C. then further concentrated to a protein content of 13.89 wt %. 85 L of the protein solution was then pasteurized at about 74° C. for 16 seconds. A 45 kg aliquot of pasteurized protein solution was spray dried to yield a product having a protein content of 91.13% (N×6.25) d.b. This product was termed MTYP64-K08-21A YP870N.

40.2 kg of the pasteurized protein solution was jet cooked at 139° C. for about 15 seconds and then spray dried to yield a product having a protein content of 92.09% (N×6.25) d.b. This product was termed MTYP64-K08-21A YP870NG.

Note that all cited pH values were from measurements conducted with the sample at room temperature unless otherwise noted.

### Example 9

48 kg of yellow split pea flour was combined with 300 L of RO water having a temperature of 53.4° C. and 25% NaOH solution to adjust the pH of the mixture. Another 48 kg of yellow split pea flour was combined with 300 L of RO water having a temperature of 52.3° C. and 25% NaOH solution to adjust the pH of the mixture. In total 0.70 kg of 25% NaOH solution was used. The mixtures were stirred for 10 minutes. A 176.03 kg portion of suspended residual solids were removed by centrifugation using a decanter centrifuge to provide a protein solution having a protein content of 3.27 wt % and a pH of 7.06. The protein solution was then heated to 86.6° C., held for at least 5 minutes then cooled to 62.7° C. and centrifuged with a disc stack centrifuge to provide 490 L of protein solution having a protein content of 2.86 wt % protein as well as 21.54 kg of finer residual solids.

The 21.54 kg of finer residual solids, having a protein content of 9.09 wt %, was combined with 86 L of RO water having a temperature of 58° C. The mixture was centrifuged with a disc stack centrifuge and 12.6 kg of washed finer residual solids and 110 L of used wash solution at 0.76 wt % protein was collected.

The protein solution was combined with the used wash solution and pH adjusted with 0.3 kg of 25% NaOH solution. The pH adjusted solution, with a pH of 7.59 and a protein content of 2.48 wt %, was reduced in volume from 600 to 180 L by concentration on a polyethersulfone membrane having a MWCO of 100,000 daltons operated at a temperature of about 54° C. The concentrated protein solution with a protein content of 8.06 wt % was then diafiltered on the same membrane with 900 L of RO water with the diafiltration operation conducted at about 54° C. then further concentrated to a protein content of 16.36 wt %. A 22.3 kg aliquot of this protein solution was pasteurized at about 72° C. for 16 seconds and then spray dried to yield a product having a protein content of 91.35% (N×6.25) d.b. This product was termed MTYP64-K29-21A YP870N.

Note that all cited pH values were from measurements conducted with the sample at room temperature unless otherwise noted. The extraction slurry was noted as having a pH of 6.93 when measured at 50.6° C.

### Example 10

48 kg of yellow split pea flour was combined with 300 L of RO water having a temperature of 53.9° C. and 25% NaOH solution to adjust the pH of the mixture. Another 48 kg of yellow split pea flour was combined with 300 L of RO water having a temperature of 54.6° C. and 25% NaOH solution to adjust the pH of the mixture. In total 0.50 kg of 25% NaOH solution was used. The mixtures were stirred for 10 minutes. A 177.6 kg portion of suspended residual solids were removed by centrifugation using a decanter centrifuge to provide a protein solution having a protein content of 3.35 wt % and a pH of 6.84. The protein solution was then heated to 66.3° C., held for a minimum of 5 minutes and then centrifuged with a disc stack centrifuge to provide 480 L of protein solution having a protein content of 2.87 wt % protein and 20.66 kg of finer residual solids.

20.66 kg of finer residual solids, having a protein content of 8.00 wt %, was combined with 85 L of RO water having a temperature of 52.6° C. The mixture was centrifuged with a disc stack centrifuge and 9.74 kg of washed finer residual solids and 100 L of used wash solution having a protein content of 0.82 wt % was collected. The washed finer residual solids, having a protein content of 6.18 wt % and a solids content of 7.59 wt % (81.42% protein (N×6.25) d.b.), was pasteurized at about 72° C. for 60 seconds and then spray dried.

The protein solution was combined with the used wash solution and the pH adjusted by the addition of 0.52 kg of 25% NaOH solution. The pH adjusted solution, having a protein content of 2.47 wt % and a pH of 7.55, was reduced in volume from 580 to 105 L by concentration on a polyethersulfone membrane having a MWCO of 100,000 daltons operated at a temperature of about 54° C. The concentrated protein solution, having a protein content of 12.87 wt %, was then diafiltered on the same membrane with 105 L of RO water with the diafiltration operation conducted at about 55° C. A 52.5 kg aliquot of the protein solution (1 volume of diafiltration) was then removed and the remaining 50 L of protein solution was diafiltered with an additional 200 L of RO water on the same membrane with the diafiltration operation conducted at about 53° C. to provide 51 kg of concentrated and diafiltered (5 volumes of diafiltration) solution.

The 52.5 kg concentrated and diafiltered (1 volume diafiltration) protein solution, having a protein content of 12.58 wt %, was pasteurized at about 72° C. for 16 seconds. A 30 kg portion of the pasteurized protein solution was then spray dried to yield a product having a protein content of 90.86% (N×6.25) d.b. This product was termed MTYP64-L06-21A YP870N-01. A 26 kg portion of the pasteurized protein solution was jet cooked at about 139-142° C. for about 45 seconds and then spray dried to yield a product having a protein content of 91.35% (N×6.25) d.b. This product was termed MTYP64-L06-21A YP870NG-01.

51 kg of concentrated and diafiltered (5 volumes diafiltration) protein solution was pasteurized at about 72° C. for 16 seconds. A 25.3 kg portion of the pasteurized protein solution was then spray dried to yield a product having a protein content of 93.71% (N x 6.25) d.b. This product was termed MTYP64-L06-21A YP870N-02. A 30.7 kg aliquot of the pasteurized protein solution was jet cooked at 140-141° C. for about 45 seconds and then spray dried to yield a product having a protein content of 92.94% (N×6.25) d.b. This product was termed MTYP64-L06-21A YP870NG-02.

Note that all cited pH values were from measurements conducted with the sample at room temperature unless otherwise noted

### Example 11

48 kg of yellow split pea flour was combined with 300 L of RO water having a temperature of 51.1° C. and 0.36 kg of 25% NaOH solution to adjust the pH of the mixture. The mixture was stirred for 10 minutes. A 78.76 kg portion of suspended residual solids were removed by centrifugation using a decanter centrifuge to provide a protein solution having a protein content of 3.67 wt % and a pH of 6.90. The protein solution was heated to 83.4-88.1° C. for at least 1 minute and then cooled to 55° C.

The protein solution, having a protein content of 3.53 wt %, was reduced in volume from 250 to 110 L by concentration on a polyethersulfone membrane having a MWCO of 100,000 daltons operated at a temperature of about 50° C. The concentrated protein solution with a protein content of 7.59 wt % was then diafiltered on the same membrane with 550 L of RO water with the diafiltration operation conducted at about 51° C. The diafiltered protein solution was then further concentrated to provide 60.94 kg of protein solution with a protein content of 13.31 wt %.

A 30.12 kg portion of the protein solution was pasteurized at about 72° C. for 16 seconds and then spray dried to yield a product having a protein content of 92.83% (N×6.25) d.b. This product was termed MTYP64-C10-22A YP870N/PN.

Another 30.82 kg portion of the protein solution was adjusted to pH 7.63 with 25% NaOH solution and then jet cooked at 140° C. for about 45 seconds. The jet cooked protein solution was spray dried to yield a product having a protein content of 92.74% (N×6.25) d.b. This product was termed MTYP64-C10-22A YP870N/PNG.

Note that all cited pH values were from measurements conducted with the sample at room temperature unless otherwise noted. The extraction slurry was noted as having a pH of 6.86 when measured at 46.1° C.

### Example 12

48 kg of yellow split pea flour was combined with 300 L of RO water having a temperature of 54.4° C. and 0.28 kg of 25% NaOH solution to adjust the pH of the mixture. The mixture was stirred for 10 minutes. A 91.12 kg portion of suspended residual solids were removed by centrifugation using a decanter centrifuge to provide a protein solution having a protein content of 2.50 wt % and a pH of 7.01. The protein solution was then heated to 75-90° C., held for a minimum of 5 minutes and then centrifuged with a disc stack centrifuge to provide 247 L of protein solution having a protein content of 1.87 wt % protein as well as 10.0 kg of finer residual solids.

The 10 kg of finer residual solids, having a protein content of 9.48 wt %, was combined with 40 L of RO water having a temperature of 50° C. The mixture was centrifuged with a disc stack centrifuge and 3.30 kg of washed finer residual solids were collected along with 56 L of used wash solution having a protein content of 1.15 wt %. The washed finer residual solids, having a protein content of 11.01 wt %, was pasteurized at about 76° C. for 5 minutes and then freeze dried to yield a product having a protein content of 88.15% (N×6.25) d.b. This product was termed MTYP69-G27-22A YP870PN

The protein solution was combined with the used wash solution and the pH of the mixture adjusted by the addition of 0.12 kg of 25% NaOH solution. The pH adjusted solution, having a protein content of 1.76 wt % and a pH of 7.76, was reduced in volume from 300 to 60 L by concentration on a polyethersulfone membrane having a MWCO of 100,000 daltons operated at a temperature of about 55° C. The concentrated protein solution with a protein content of 7.40 wt % was then diafiltered on the same membrane with 240 L of RO water with the diafiltration operation conducted at about 53° C., then further concentrated to a protein content of 12.38 wt %.

The diafiltered protein solution was pasteurized at about 72° C. for 60 seconds and then spray dried to yield a product having a protein content of 85.17% (N×6.25) d.b. This product was termed MTYP69-G27-22A YP870N.

Note that all cited pH values were from measurements conducted with the sample at room temperature unless otherwise noted. The extraction slurry was noted as having a pH of 6.87 when measured at 49.3° C.

### Example 13

48 kg of yellow split pea flour was combined with 300 L of RO water having a temperature of 55° C. and 0.24 kg of 25% NaOH solution to adjust the pH of the mixture. The mixture was stirred for 10 minutes. A 90.87 kg portion of suspended residual solids were removed by centrifugation using a decanter centrifuge to provide a protein solution having a protein content of 2.69 wt % and a pH of 7.03. The protein solution was heated to 75° C., held for a minimum of 5 minutes and then centrifuged with a disc stack centrifuge to provide 255 L of protein solution having a protein content of 1.82 wt % protein as well as 9.1 kg of finer residual solids.

9.1 kg of finer residual solids, having a protein content of 13.09 wt %, was combined with 50 L of RO water having a temperature of 3° C. The mixture was centrifuged with a disc stack centrifuge and 4.2 kg of washed finer residual solids was collected along with 60 L of wash solution having a protein content of 1.17 wt % protein. The washed finer residual solids were freeze dried to yield a product having a protein content of 88.52% (N×6.25) d.b. This product was termed MTYP69-G28-22A YP870PN

The protein solution was pH adjusted by the addition of 0.12 kg of 25% NaOH solution. 255 L of the pH adjusted protein solution, having a pH of 7.57, was reduced in volume to 55 L by concentration on a polyethersulfone membrane having a MWCO of 100,000 daltons, operated at a temperature of about 56° C. The concentrated protein solution, having a protein content of 6.68 wt %, was then diafiltered on the same membrane with 220 L of RO water with the diafiltration operation conducted at a temperature of about 53° C., then further concentrated to a protein content of 10.16 wt %.

34.2 kg of diafiltered protein solution was pasteurized at about 73° C. for 60 seconds and then spray dried to yield a product having a protein content of 85.33% (N×6.25) d.b. This product was termed MTYP69-G28-22A YP870N.

Note that all cited pH values were from measurements conducted with the sample at room temperature unless otherwise noted. The extraction slurry was noted as having a pH of 6.87 when measured at 50.2° C.

### Example 14

42 kg of raw fava bean flour was combined with 300 L of RO water having a temperature of 42.3° C., 0.74 kg of 25% NaOH solution and 0.15 kg of ascorbic acid. Another 42 kg of raw fava bean flour was combined with 300 L of RO water having a temperature of 43.9° C., 0.74 kg of 25% NaOH solution and 0.15 kg of ascorbic acid. The mixtures were stirred for 30 minutes. A portion of the suspended solids (115.25 kg) were removed by centrifugation using a decanter centrifuge to provide a protein solution having a protein content of 4.74 wt % and a pH of 7.52. The protein solution was then further clarified by centrifugation using a disc stack centrifuge to remove finer residual solids (19.89 kg) and provide 510L of protein solution having a protein content of 4.49 wt %. The finer residual solids were combined with about 100 L of RO water of unrecorded temperature (target of 50° C.) and mixed for an unrecorded time (target of 5 minutes). The mixture was then centrifuged with a disk stack centrifuge to provide 19.18 kg of washed finer residual solids having a protein content of 4.91 wt % and a used wash solution having a protein content of 0.79 wt %.

19.18 kg of washed finer residual solids were pasteurized at about 75° C. for about 60 seconds. 18.62 kg of pasteurized washed finer residual solids were spray dried to yield a product having a protein content of 82.42% (N×6.25) d.b. The product was termed FV02-C14-22A FV870PN.

The protein solution was combined with the used wash solution. 610 L of the combined solution was then reduced in volume by concentration on a polyethersulfone membrane having a pore size of 10,000 daltons, operated at a temperature of about 44° C. During the concentration step 240 L of retentate was removed and discarded to reduce the volume of material to be processed. The remaining protein solution was further reduced in volume to 120 L with the concentration step operated at a temperature of about 30° C. The concentrated protein solution, having a protein content of 7.40 wt %, was then diafiltered on the same membrane with 120 L of RO water, with the diafiltration operation conducted at about 30° C., then further concentrated to a protein content of 20.56 wt %. 39.80 kg of diafiltered and concentrated protein solution was pasteurized at about 75° C. for 30 seconds. 36.10 kg of pasteurized solution was diluted with 8.0 L of RO water then spray dried to yield a product having a protein content of 94.43 wt % (N×6.25) d.b. The product was termed FV02-C14-22A FV870N.

Note all cited pH values were from measurements conducted with the sample at room temperature unless otherwise noted.

### Example 15

30 kg of decorticated yellow lentil flour (60 mesh) was combined with 300 L of tap water having a temperature of 21.9° C. and 0.28 kg of 25% NaOH solution. The mixture was stirred for 35 minutes. A portion of the suspended solids (41.18 kg) were removed by centrifugation using a decanter centrifuge to provide a protein solution having a protein content of 2.53 wt % and a pH of 7.22. The protein solution was then reduced in volume from 270 L to 70 L by concentration on a polyethersulfone membrane having a molecular weight cutoff of 100,000 daltons, operated at a temperature of about 21° C. The protein solution, with a protein content of 8.86 wt %, was then diafiltered on the same membrane with 490 L of RO water with the diafiltration operation conducted at about 20° C. The diafiltered protein solution had a protein content of 8.76 wt % and a pH of 7.17. 62.06 kg of this protein solution was pasteurized at about 80° C. for 60 seconds. 60.93 kg of pasteurized solution was spray dried to yield a product having a protein content of 90.91 wt % (N×6.25) d.b. The product was termed LE04-C22-22A LE870N/PN.

### Example 16

This Example illustrates the protein solubility of the pulse protein products prepared as described in Examples 1 to 14.

A 100 ml beaker and magnetic stir bar were pre-weighed. Sufficient protein powder to supply 2 g of protein was weighed into the beaker. 10-15 ml of RO water was added and the sample stirred with the stir bar until the powder was thoroughly wetted. At this point another 25-30 ml of RO water was added and mixed in. The pH of the sample was adjusted to the target value with 0.5M NaOH or HCl as necessary and the sample stirred on a magnetic stir plate set to a speed just below forming a vortex in the sample for about 55-60 minutes with the pH periodically checked and adjusted if necessary during this time. At the end of the stirring time the pH of the sample was checked and corrected as necessary again and then additional RO water added to bring the sample weight to 50 g (protein concentration of 4% w/w) and mixed in. Approximately 20 ml of the dispersion was then transferred to a 50 ml centrifuge tube and centrifuged at 10,000 rpm (7,800 g) in a Sorvall SS-34 rotor for 10 minutes with the centrifuge set to 20° C. After the centrifugation was completed 10 ml of supernatant was removed from the centrifuge tube by pipet. Samples of the supernatant and the original dispersion were tested for protein content by combustion analysis (N×6.25).

Solubility (%)=(supernatant protein conc./original dispersion protein conc.)×100

The protein solubility of the pulse protein products prepared as described in Examples 1 to 14 are shown in Table 3.

TABLE 3
Solubility of pulse protein products at different pH values

| | Solubility (%) | | |
|---|---|---|---|
| Product | pH 4 | pH 5.5 | pH 7 |
| MTYP61-F29-21A YP870N | 22.5 | 14.2 | 54.7 |
| MTYP62-H26-21A YP870N | 25.9 | 11.8 | 33.3 |
| MTYP62-I02-21A YP870N | 25.6 | 11.0 | 33.3 |
| MTYP62-I08-21A YP870N | 37.4 | 11.4 | 20.3 |
| MTYP62-I09-21A YP870N | 32.4 | 12.5 | 34.3 |
| MTYP63-I29-21A YP870N | 26.7 | 13.1 | 67.9 |
| MTYP64-J07-21A YP870N | 54.6 | 11.7 | 50.8 |
| MTYP63-J13-21A YP870N | 27.5 | 13.0 | 62.6 |
| MTYP64-J14-21A YP870N | 52.5 | 11.5 | 53.7 |
| MTYP64-K08-21A YP870N | 51.4 | 11.3 | 64.6 |
| MTYP64-K29-21A YP870N | 36.2 | 10.3 | 68.1 |
| MTYP64-L06-21A YP870N-01 | 43.8 | 5.8 | 50.6 |
| MTYP64-C10-22A YP870N/PN | 29.7 | 11.7 | 44.6 |
| MTYP69-G27-22A YP870N | 42.1 | 11.9 | 46.5 |
| MTYP69-G28-22A YP870N | 43.2 | 20.4 | 52.6 |
| FV02-C14-22A FV870N | 9.1 | 5.3 | 76.7 |
| MTYP64-K08-21A YP870NG | 44.8 | 11.1 | 59.9 |
| MTYP64-L06-21A YP870NG-01 | 50.5 | 9.5 | 63.5 |
| MTYP64-C10-22A YP870N/PNG | 24.3 | 5.6 | 72.2 |
| MTYP64-J14-21A YP870N + PN | 51.0 | 12.0 | 42.4 |
| MTYP62-H26-21A YP870PN | 16.1 | 8.0 | 33.8 |
| MTYP62-I08-21A YP870PN | 20.2 | 10.1 | 34.7 |
| MTYP62-I09-21A YP870PN | 10.5 | 7.6 | 36.2 |
| MTYP63-I29-21A YP870PN | 11.6 | 4.4 | 17.7 |
| MTYP64-K08-21A YP870PN-01 | 10.0 | 8.8 | 19.4 |
| MTYP64-K08-21A YP870PN-02 | 6.6 | 3.2 | 7.0 |
| FV02-C14-22A FV870PN | 16.6 | 0.7 | 16.5 |

As may be seen from the results presented in Table 3, when yellow pea was the protein source the solubility of the product of the invention derived from the solution of soluble protein (870N) was higher in solubility than the corresponding product of the invention derived from the finer solids (870PN) at pH 4.

### Example 17

This Example contains an evaluation of the dry colour of the pulse protein products prepared as described in Examples 1 to 15. Dry colour (CIE L*a*b*) was assessed using a HunterLab ColorQuest XE instrument operated in reflectance mode (RSEX) with an illuminant setting of D65 and an observer setting of 10°. The results are shown in the following Table 4.

TABLE 4
Dry colour of pulse protein products

| Product | L* | a* | b* |
|---|---|---|---|
| MTYP61-F29-21A YP870N | 82.83 | 1.41 | 21.66 |
| MTYP62-H26-21A YP870N | 81.11 | 1.97 | 21.49 |
| MTYP62-I02-21A YP870N | 80.70 | 1.74 | 25.17 |
| MTYP62-I08-21A YP870N | 80.60 | 1.81 | 23.08 |
| MTYP62-I09-21A YP870N | 80.47 | 2.03 | 25.99 |
| MTYP63-I29-21A YP870N | 81.26 | 1.01 | 18.42 |
| MTYP64-J07-21A YP870N | 82.71 | 0.68 | 16.55 |
| MTYP63-J13-21A YP870N | 81.39 | 1.18 | 19.55 |
| MTYP64-J14-21A YP870N | 81.10 | 1.01 | 17.68 |
| MTYP64-K08-21A YP870N | 81.09 | 1.12 | 17.01 |
| MTYP64-K29-21A YP870N | 80.71 | 1.03 | 19.13 |
| MTYP64-L06-21A YP870N-01 | 80.79 | 1.44 | 17.46 |
| MTYP64-L06-21A YP870N-02 | 81.12 | 1.06 | 16.40 |
| MTYP64-C10-22A YP870N/PN | 81.20 | 0.91 | 17.40 |
| MTYP69-G27-22A YP870N | 77.70 | 1.15 | 23.43 |
| MTYP69-G28-22A YP870N | 78.00 | 1.15 | 22.17 |
| FV02-C14-22A FV870N | 83.72 | −0.44 | 17.04 |
| LE04-C22-22A LE870N/PN | 97.99 | 0.86 | 14.34 |
| MTYP64-K08-21A YP870NG | 80.81 | 0.88 | 16.52 |
| MTYP64-L06-21A YP870NG-01 | 80.79 | 1.04 | 16.22 |
| MTYP64-L06-21A YP870NG-02 | 79.41 | 1.12 | 17.48 |
| MTYP64-C10-22A YP870N/PNG | 80.85 | 0.58 | 16.43 |
| MTYP64-J14-21A YP870N + PN | 81.01 | 0.96 | 17.66 |
| MTYP62-H26-21A YP870PN | 79.36 | 1.89 | 26.32 |
| MTYP62-I02-21A YP870PN | 79.29 | 1.02 | 22.86 |
| MTYP62-I08-21A YP870PN | 80.08 | 1.30 | 22.72 |
| MTYP62-I09-21A YP870PN | 78.19 | 1.08 | 24.61 |
| MTYP63-I29-21A YP870PN | 81.58 | 0.77 | 18.83 |
| MTYP64-K08-21A YP870PN-01 | 81.23 | 1.16 | 19.96 |
| MTYP64-K08-21A YP870PN-02 | 80.27 | 0.65 | 18.03 |
| FV02-C14-22A FV870PN | 81.07 | −0.38 | 18.53 |

As may be seen from the results presented in Table 4, the product of the invention derived from the solution of soluble protein (870N) generally had a similar colour to the product of the invention derived from the finer solids (870PN).

### Example 18

This Example contains an evaluation of the water binding capacity of the pulse protein products prepared as described in Examples 1, 3-9, 11 and 14-15.

The water binding capacity of the products was determined by the following procedure. Protein powder (1 g) was weighed into centrifuge tubes (50 ml) of known weight. To this powder was added approximately 20 ml of RO water at the natural pH. The contents of the tubes were mixed using a vortex mixer at moderate speed for 1 minute. The samples were incubated at room temperature for 5 minutes then mixed with the vortex for 30 seconds. This was followed by incubation at room temperature for another 5 minutes then another 30 seconds of vortex mixing. The samples were then centrifuged at 1,000 g for 15 minutes at 20° C. After centrifugation, the supernatant was carefully poured off, ensuring that all solid material remained in the tube. The centrifuge tube was then re-weighed and the weight of water saturated sample was determined.

Water binding capacity (WBC) was calculated as:

WBC (ml/g)=(mass of water saturated sample (g)−mass of initial sample (g))/(mass of initial sample (g)×total solids content of sample).

The WBC results are shown in Table 5.

TABLE 5
WBC of pulse protein products

| Product | WBC (ml/g) |
|---|---|
| MTYP62-H26-21A YP870N | 3.25 |
| MTYP62-I08-21A YP870N | 2.57 |
| MTYP62-I09-21A YP870N | 3.38 |
| MTYP63-I29-21A YP870N | 0.31 |
| MTYP64-J07-21A YP870N | 1.97 |
| MTYP63-J13-21A YP870N | 3.22 |
| MTYP64-J14-21A YP870N | 1.21 |
| MTYP64-K08-21A YP870N | 1.82 |
| MTYP64-K29-21A YP870N | 5.23 |
| MTYP64-C10-22A YP870N/PN | 4.31 |
| FV02-C14-22A FV870N | 1.65 |
| LE04-C22-22A LE870N/PN | 4.19 |
| MTYP64-J14-21A YP870N + PN | 2.32 |
| MTYP64-K08-21A YP870NG | 0.93 |
| MTYP62-H26-21A YP870PN | 2.94 |
| MTYP62-I08-21A YP870PN | 2.60 |
| MTYP62-I09-21A YP870PN | 2.82 |
| MTYP63-I29-21A YP870PN | 3.48 |
| FV02-C14-22A FV870PN | 3.88 |

As may be seen from the results in Table 5, there was a broad range of water binding capacities determined for the product of the invention derived from the solution of soluble protein (870N). The range of values determined for the finer solids derived product was narrower but were within the range of the 870N values.

### Example 19

This Example contains an evaluation of the oil binding capacity of the pulse protein products prepared as described in Examples 1, 3-9, 11 and 14-15.

The oil binding capacity of the products was determined by the following procedure. Protein powder (1 g) was weighed into centrifuge tubes (50 ml) of known weight. To this powder was added approximately 20 ml of retail canola oil. The contents of the tubes were mixed using a vortex mixer at moderate speed for 1 minute. The samples were incubated at room temperature for 5 minutes then mixed with the vortex for 30 seconds. This was followed by incubation at room temperature for another 5 minutes then another 30 seconds of vortex mixing. The samples were then centrifuged at 1,000 g for 15 minutes at 20° C. After centrifugation, the supernatant was pipetted off, ensuring that all solid material remained in the tube. The centrifuge tube was then re-weighed and the weight of oil saturated sample was determined.

Oil binding capacity (OBC) was calculated as:

OBC (ml/g)=((mass of oil saturated sample (g)−mass of initial sample (g))/0.914 g/ml)/(mass of initial sample (g)×total solids content of sample).

The OBC results are shown in Table 6.

TABLE 6
OBC of pulse protein products

| Product | OBC (ml/g) |
|---|---|
| MTYP62-H26-21A YP870N | 1.36 |
| MTYP62-I08-21A YP870N | 1.03 |
| MTYP63-I29-21A YP870N | 2.09 |
| MTYP64-J07-21A YP870N | 1.42 |
| MTYP63-J13-21A YP870N | 1.38 |
| MTYP64-J14-21A YP870N | 1.38 |
| MTYP64-K08-21A YP870N | 1.40 |
| MTYP64-K29-21A YP870N | 1.01 |
| MTYP64-C10-22A YP870N/PN | 1.27 |
| FV02-C14-22A FV870N | 1.55 |
| LE04-C22-22A LE870N/PN | 0.87 |
| MTYP64-J14-21A YP870N + PN | 1.33 |
| MTYP64-K08-21A YP870NG | 1.32 |
| MTYP62-H26-21A YP870PN | 1.44 |
| MTYP62-I08-21A YP870PN | 1.67 |
| MTYP62-I09-21A YP870PN | 1.66 |
| MTYP63-I29-21A YP870PN | 0.86 |
| FV02-C14-22A FV870PN | 1.13 |

As may be seen from the results in Table 6, the oil binding capacities determined for the product of the invention derived from the solution of soluble protein (870N) were similar to the those determined for the product of the invention derived from the finer solids (870PN).

### Example 20

This Example contains an evaluation of the phytic acid content of the pulse protein products prepared as described in Examples 1 to 3. Phytic acid content was determined using the method of Latta and Eskin (J. Agric. Food Chem., 28:1313-1315). Results are shown in Table 7 below.

TABLE 7
Phytic acid content of pulse protein products

| Product | % phytic acid (d.b.) |
|---|---|
| MTYP61-F29-21A YP870N | 1.83 |
| MTYP62-H26-21A YP870N | 1.49 |
| MTYP62-I02-21A YP870N | 1.28 |
| MTYP62-I08-21A YP870N | 1.08 |
| MTYP62-H26-21A YP870PN | 1.15 |
| MTYP62-I02-21A YP870PN | 2.71 |
| MTYP62-I08-21A YP870PN | 1.65 |

As may be seen from the results in Table 7, there generally did not appear to be much difference in the phytic acid content between the product of the invention derived from the solution of soluble protein (870N) and the product of the invention derived from the finer solids (870PN).

### Example 21

This Example illustrates the sodium content of products prepared as described in Examples 3, 6, 11, 12 and 15.

Sodium contents were determined by an ICP-OES method by Central Testing Laboratory Ltd. (Winnipeg, MB). Results are shown in Table 8 below.

TABLE 8
Sodium content of pulse protein products

| Product | % sodium (d.b.) |
|---|---|
| MTYP62-I08-21A YP870N | 0.08 |
| MTYP62-I09-21A YP870N | 0.12 |
| MTYP63-J13-21A YP870N | 0.10 |
| MTYP64-C10-22A YP870N/PN | 0.06 |
| MTYP69-G27-22A YP870N | 0.12 |
| LE04-C22-22A LE870N/PN | 0.10 |

As may be seen from the results in Table 8, all of the products tested were low in sodium.

### Example 22

This Example describes the Amino Acid profile of the pulse protein products prepared as described in Example 3.

Amino acid profile of the pulse protein product was assessed experimentally (USDA MSS2 (1993)) by Merieux NutriSciences (Crete, IL). A complete amino acid profile analysis was done, to quantify tryptophan, cysteine/methionine and the remaining amino acids.

The amino acid profile for the pulse protein products prepared as described in Example 3 is shown in Table 9.

TABLE 9
Amino acid profile of protein products

| | concentration (mg/g protein) | |
|---|---|---|
| amino acid | YP62-I08-21A YP870N | YP62-I08-21A YP870PN |
| Aspartic | 127.4 | 125.4 |
| Threonine | 45.6 | 43.6 |
| Serine | 54.3 | 54.2 |
| Glutamic | 191.7 | 189.8 |
| Proline | 50.4 | 50.3 |
| Glycine | 45.1 | 45.3 |
| Alanine | 51.0 | 49.2 |
| Valine | 50.5 | 50.8 |
| Isoleucine | 48.4 | 47.5 |
| Leucine | 83.5 | 86.3 |
| Tyrosine | 43.7 | 41.6 |
| Phenylalanine | 54.8 | 56.6 |
| Lysine | 81.8 | 80.0 |
| Histidine | 27.9 | 27.9 |
| Arginine | 90.8 | 92.4 |
| Cysteine | 13.4 | 14.7 |
| Methionine | 11.1 | 11.5 |
| Tryptophan | 8.5 | 9.0 |

As may be seen from the results presented in Table 9, amino acid profiles of the products tested were similar.

### Example 23

This Example illustrates the viscosity in solution of the pulse protein products prepared as described in Examples 1-3, 7, 9 and 15. Solutions of the products were prepared at 20% protein and the viscosity of the solutions determined at different shear rates using an

Anton Paar MCR 302 rheometer fitted with a PP25 plate/plate system. The protein solution sample was placed on the bottom plate and the upper plate was lowered to a 1 mm gap. The viscosity was tested at 25° C. with increasing shear rate from 0.1 to 100 1/s.

Results are as shown in Table 10.

TABLE 10
Viscosity of pulse protein solutions at different shear rates

| | Viscosity (mPa · s) | | | | | |
|---|---|---|---|---|---|---|
| Shear | MTYP62- | MTYP62- | MTYP- | MTYP64- | MTYP64- | LE04- |
| rate | H26-21A | I02-21A | I08-21A | J14-21A | K29-21A | C22-22A |
| (1/s) | YP870N | YP870N | YP870N | YP870N | YP870N | LE870N/PN |
| 1.28 | 375,806 | 427,626 | 88,024 | 85,923 | 294,678 | 1,146,231 |
| 1.84 | 263,419 | 275,745 | 59,189 | 63,736 | 209,179 | 752,201 |
| 2.64 | 183,954 | 167,814 | 40,190 | 47,140 | 147,788 | 500,296 |
| 3.79 | 126,499 | 99,442 | 28,525 | 34,904 | 102,070 | 324,975 |
| 5.46 | 89,572 | 60,177 | 20,486 | 25,388 | 75,433 | 218,716 |
| 7.86 | 63,168 | 37,695 | 14,891 | 18,656 | 54,233 | 139,394 |
| 11.30 | 44,631 | 27,975 | 20,927 | 13,821 | 37,935 | 90,586 |
| 16.30 | 31,297 | 19,130 | 7,992 | 10,233 | 26,583 | 79,868 |
| 23.40 | 20,878 | 13,928 | 5,828 | 7,398 | 16,982 | 60,573 |
| 33.60 | 14,493 | 11,167 | 4,385 | 5,341 | 11,715 | 43,905 |
| 48.40 | 9,257 | 8,427 | 3,439 | 3,838 | 7,554 | 25,894 |
| 69.60 | 6,122 | 7,673 | 2,861 | 2,788 | 5,150 | 13,237 |
| 100.00 | 4,212 | 6,389 | 2,363 | 2,094 | 3,733 | 8,815 |

As may be seen from the results in Table 10, the product prepared from yellow lentil had a higher viscosity than the product prepared from yellow pea.

### Example 24

This Example illustrates the preparation and sensory assessment of chocolate chip oatmeal cookies containing 4% protein from the pulse protein product prepared by the procedure of Example 1.

The formulation for the cookies is shown in Table 11:

TABLE 11
Formulation for cookies with pulse protein product of the invention

| Ingredient | Amount (g) | Percentage (%) |
|---|---|---|
| Butter | 111.64 | 13.28 |
| Canola oil | 5.01 | 0.60 |
| Packed brown sugar | 104.38 | 12.42 |
| White sugar | 54.14 | 6.44 |
| MTYP-F29-21A YP870N | 37.75 | 4.49 |
| Water, RO | 37.45 | 4.46 |
| Oatmilk | 15.54 | 1.85 |
| Vanilla | 4.68 | 0.56 |
| Flour | 136.1 | 16.19 |
| Baking soda | 2.84 | 0.34 |
| Salt | 1.65 | 0.20 |
| Oats | 132.4 | 15.75 |
| Chocolate chips | 197.01 | 23.44 |
| Total | 840.59 | 100.00 |

The butter, oil, white sugar and brown sugar were placed in a Hobart stand mixer and creamed together. The protein powder and water were manually mixed in a beaker then added to the creamed mixture in the Hobart. The oatmilk and vanilla were then added. The flour, baking soda, salt, and oats were mixed in a bowl then slowly added to the ingredients in the Hobart while mixing on low-medium speed. Once incorporated, the chocolate chips were added either on low speed in the Hobart or mixed in by hand. The cookie batter was dropped by spoonful onto a baking pan and baked at 350° F. for 7-9 min or until slightly golden brown.

The cookies were tasted by an informal sensory panel with 11 participants. Comments provided by the panelists indicated that an edible cookie of at least acceptable quality was prepared with the product of the invention. Specific comments included “Smells like normal cookie”, “Good chocolate aroma”, “Good aroma”, “No off aroma”, “Good smell”, “No off odour”, “No aroma”, “Crispy outside”, “Soft inside”, “Chewy”, “Crunchy mouthfeel”, “A bit dry”, “A bit hard”, “Good chewiness”, Great texture”, “Not that soft”, “A bit dry on top and bottom” “No spread”, “Looks dry”, “Interior is darker colour, more moist?”, “Tastes like a regular chocolate chip cookie”, “Not too sweet”, “A bit salty”, “Delicious”, “Good flavour”, “Great flavour”, “Didn't notice any off taste”, “Tastes very good”, “Not very sweet”, “No off flavour”, “Not very tasty”.

It will be appreciated that the term “about” applies to all reported measurements and calculations. Such a term accounts for instrumental and measurement error as would be appreciated and comprehended by those in the intended field.

It will be appreciated that the examples and embodiments disclosed herein are intended to be non-limiting. They are illustrative of the invention and may be modified or altered. Such modifications and alterations within the concept and spirit of the intended invention.

We claim:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A process for preparing a pulse protein product from a pulse protein source, the pulse protein product having a protein content of greater than 60 wt % (N×6.25) d.b., the process comprising:
g) extracting pulse protein from a pulse protein source to cause solubilization of the pulse protein from the pulse protein source to produce an aqueous phase and a residual pulse protein source, optionally wherein a pH of the extraction may be the natural pH of the combination of the water and the pulse protein source, or the pH of the extraction may be adjusted up to any value between the natural pH and about 8.0, or the pH may be adjusted within the range of about 6.8 to about 8.0, or preferably the pH may be adjusted to about 6.8 to about 7.5,
h) separating the aqueous phase from the residual pulse protein source to produce an aqueous pulse protein solution and a separated residual pulse protein source, optionally by centrifugation and/or filtration, optionally with a decanter centrifuge and a disc stack centrifuge,
i) optionally adjusting the pH of the aqueous pulse protein solution to a higher value not exceeding about pH 8.0;
j) optionally concentrating the aqueous pulse protein solution to provide a concentrated pulse protein solution having a protein concentration of about 5 to about 30 wt %, preferably about 5 to about 20 wt %, more preferably about 10 to about 20 wt %;
k) optionally diafiltering the concentrated pulse protein solution using water as the diafiltration solution OR diafiltering the aqueous pulse protein solution prior to concentration or diafiltering the partially concentrated aqueous pulse protein solution;
l. optionally drying the pulse protein solution or optionally concentrated and/or optionally diafiltered pulse protein solution, optionally by any conventional means such as spray drying or freeze drying, to provide a pulse protein product;
wherein steps d) and c) may optionally be carried out simultaneously.

78. The process of claim 77 further comprising:
treating the aqueous pulse protein solution with an anti-foamer, such as any suitable food-grade, non-silicone based anti-foamer, to reduce the volume of foam formed upon further processing and wherein the anti-foamer is optionally added during the extraction step a), wherein optionally the quantity of anti-foamer employed is generally greater than about 0.0003% w/v.

79. The process of claim 77, further comprising:
treating the aqueous pulse protein solution with an adsorbent, such as granulated activated carbon, to remove colour and/or odour compounds.

80. The process of claim 77, further comprising the step of defatting the separated aqueous pulse protein solution to at least partially remove fat from the separated aqueous pulse protein solution, optionally by centrifugation and/or filtration.

81. The process of claim 77, wherein solubilization of the protein is effected at a temperature of from about 1° to about 100° C., preferably about 15° to about 65° C., more preferably about 20° C. to about 35° C. or about 50° to about 60° C., preferably accompanied by agitation, and/or the solubilization time is about 1 to about 60 minutes, preferably about 10 to about 30 minutes.

82. The process of claim 77, wherein the water of extraction may contain an antioxidant, such as ascorbic acid, optionally in an amount of from about 0.01 to about 1 wt % of the solution, preferably about 0.05 to about 0.15 wt %.

83. The process of claim 77, wherein the concentration step d) and/or the diafiltration step e) are effected in such a manner that the pulse protein product subsequently recovered contains at least about 60 wt %, at least about 65 wt %, at least about 70 wt %, at least about 75 wt %, at least about 80 wt %, at least about 85 wt %, or greater than about 90 wt % protein (N×6.25) d.b.

84. The process of claim 77, wherein the optionally concentrated and optionally diafiltered aqueous pulse protein solution is pasteurized prior to optional drying or further processing and wherein pasteurization optionally comprises heating the optionally concentrated and optionally diafiltered pulse protein solution to a temperature of about 55° to about 75° C. for about 15 seconds to about 60 minutes or about 55° to about 85° C. for about 10 seconds to about 60 minutes, preferably about 60° to about 70° C. for about 10 minutes to about 60 minutes or about 70° to about 85° C. for about 10 seconds to about 60 seconds, and optionally the pasteurized pulse protein solution is cooled, such as to a temperature of about 20° to about 35° C.

85. The process of claim 77, wherein the optionally concentrated and optionally diafiltered aqueous pulse protein solution is jet cooked prior to optional drying to a temperature of about 110° C. to about 150° C. for about 10 seconds to about 1 minute, preferably to about 135° C. to 145° C. for about 40 to 50 seconds.

86. The process of claim 77, wherein when the separation step b) is done in more than one step (e.g. employing decanter centrifuge then disc stack centrifuge such as defined in claim 1b), different temperatures may be employed for each step of the separation process, preferably about 20 to about 60° C., more preferably about 20 to about 35° C. or about 50 to about 60° C. for the initial separation step (decanter centrifuge) and about 50 to about 85° C., more preferably about 50 to about 80° C., most preferably about 50 to about 60° C. for the second separation step (disc stack centrifuge), and optionally wherein the temperature of the protein solution is raised after the first separation step and held for at least 1 minute, preferably at least 5 minutes, before the second separation step, optionally to about 65° to about 85° C., preferably about 65° to about 80° C., and optionally wherein the temperature of the protein solution is raised after the first separation step and subsequently lowered before the second separation step optionally to about 50° to about 60° C. before the second separation step.

87. The process of claim 77, further comprising the step of:
bi) optionally further processing the separated residual pulse protein source obtained in step b) such as to recover residual protein; or
bii) optionally re-extracting the separated residual pulse protein source obtained in step b) with fresh water to recover residual protein and separating the re-extraction protein solution from the residual pulse protein source and optionally combining the re-extraction protein solution with the aqueous pulse protein solution for further processing; or
biii) using the separated residual pulse protein source obtained in step b) in food products, pet foods, animal feed and in industrial, cosmetic and personal care products; or
biii) drying the separated residual pulse protein source obtained in step b) for disposal or for future use in food products, pet foods, animal feed and in industrial, cosmetic and personal care products.

88. The process of claim 77, wherein extraction step a) is carried out using a counter-current extraction procedure.

89. The process of claim 77, wherein the finer solids are captured separately from the bulk of the separated residual pulse protein source in step b), optionally by the disc stack centrifuge, are optionally diluted with water, optionally RO water, then optionally dried to form a pulse protein product having a protein content of at least about 50 wt % (N×6.25) d.b., preferably at least about 55, 60, 65. 70 and 75 wt % (N×6.25) d.b., more preferably at least about 80 wt % (N×6.25) d.b, optionally wherein the pH of the optionally diluted finer solids is raised to a value between the natural pH of the solids and a pH of about 8.0, by any conventional means such as by the addition of sodium hydroxide, potassium hydroxide or any other conventional food grade alkali or combination thereof prior to optional drying to form a pulse protein product having a protein content of at least about 50 w1% (N×6.25) d.b., preferably at least about 55, 60, 65, 70 and 75 wt % (N×6.25) d.b., more preferably at least about 80 wt % (N×6.25) d.b., or wherein the finer solids captured separately from the bulk of the separated residual pulse protein source in step b), optionally by the disc stack centrifuge, are washed in order to remove contaminants and improve the purity and flavour of the product, optionally by suspending the solids in between about 1 and about 20 volumes, preferably about 1 to about 10 volumes, more preferably between about 1 and 5 volumes of wash solution such as water, preferably RO water, and optionally the washing step is conducted at any conventional temperature such as about 1° to about 75° C., preferably about 15° to about 65° C., more preferably about 50° to about 60° C., and optionally for any conventional length of time, preferably 15 minutes or less, then the washed finer solids are separated from the used wash solution by any convenient means such as centrifugation using a disc stack centrifuge, and wherein the used wash solution separated from the finer solids is optionally added to the aqueous pulse protein solution arising from the separation step b) for further processing, and optionally. wherein the washed finer solids are optionally diluted with water then optionally dried by any conventional means such as spray drying or freeze drying to provide a pulse protein product having a protein content of at least about 50 wt % (N×6.25) d.b., preferably at least about 55, 60, 65, 70, 75, 80 and 85 wt % (N×6.25) d.b., more preferably about 90 wt % (N×6.25) d.b., and optionally wherein the pH of the optionally diluted washed finer solids is adjusted to a value between the natural pH of the mixture of finer solids and water and about 8.0, by any conventional means such as by the addition of sodium hydroxide, potassium hydroxide or any other conventional food grade alkali and combinations thereof, prior to optional drying or the finer solids are pH adjusted during the washing step by adjusting the mixture of finer solids and wash solution to a pH between the natural pH of the mixture and about 8.0 using food grade alkali, then separating the washed solids from the used wash solution by centrifugation and optionally diluting and optionally drying the washed solids.

90. The process of claim 89, further comprising pasteurizing the optionally diluted and optionally pH adjusted finer solids or optionally diluted and optionally pH adjusted washed finer solids prior to the optional drying step, wherein pasteurization optionally comprises heating to a temperature of about 55° to about 75° C. for about 15 seconds to about 60 minutes or about 55° to about 85° C. for about 10 seconds to about 60 minutes, preferably about 60° to about 70° C. for about 10 minutes to about 60 minutes or about 70° to about 85° C. for about 10 seconds to about 60 seconds.

91. A pulse protein product defined as:
having a sodium content of less than about 0.40% d.b. or preferably less than about 0.30% d.b. more preferably less than about 0.12% d.b.;
having viscosity for a 20 wt % protein solution of less than about 10,000 mPa·s when measured at a shear rate of 100 (1/s), preferably the viscosity is less than about 6,389 mPa·s when measured at a shear rate of 100 (1/s);
having a solubility of less than about 60% when measured at pH 4, less than about 25% when measured at pH 5.5 and less than about 80% when measured at pH 7, optionally having a solubility of about 6.6 to about 20.2% when measured at pH 4, about 0.7 to about 10.1% when measured at pH 5.5 and about 7.0 to about 36.2% when measured at pH 7; optionally having a solubility of about 9.1 to about 54.6% when measured at pH 4, about 5.3 to about 20.4% when measured at pH 5.5 and about 20.3 to about 76.7% when measured at pH 7, optionally having a solubility of about 22.5 to about 54.6% when measured at pH 4, about 5.8 to about 20.4% when measured at pH 5.5 and about 20.3 to about 68.1% when measured at pH 7; or
having a dry colour L* value of about 77.70 to about 97.99, a dry colour a* value of about −0.44 to about 2.03 and a dry colour b* value of about 14.34 to about 26.32, preferably wherein the dry colour L* value is about 77.70 to about 82.83, the dry colour a* value is about 0.58 to about 2.03 and the dry colour b* value is about 16.40 to about 26.32; and
optionally wherein the pulse protein product is derived from a pulse source selected from lentils, chickpeas, dry peas, dry beans, lupines, bambara beans, broad or fava beans, dry pigeon peas, vetches, dried cowpeas, winged beans, sword beans and yellow peas and wherein the pulse source is optionally dehulled.

92. A pulse protein product or a residual pulse protein product having an attribute from one or more of the following tables:
a solubility as defined in or captured by Table 3,
a dry colour as defined in or captured by Table 4;
a water binding capacity as defined or captured by Table 5;
an oil binding capacity as defined in or captured by Table 6;
a phytic acid content as defined in or captured by Table 7;
a sodium content as defined in or captured by Table 8;
an amino acid profile comprising one or more amino acids as defined or captured by Table 9;
a viscosity for a 20 wt % protein solution as defined in or captured by Table 10; and
optionally wherein the pulse protein product or residual pulse protein product is derived from a pulse source selected from lentils, chickpeas, dry peas, dry beans, lupines, bambara beans, broad or fava beans, dry pigeon peas, vetches, dried cowpeas, winged beans, sword beans and yellow peas and wherein the pulse source is optionally dehulled.

93. A food or beverage comprising a pulse protein product which is defined as:
having a sodium content of less than about 0.40% d.b. or preferably less than about 0.30% d.b. more preferably less than about 0.12% d.b.;
having viscosity for a 20 wt % protein solution of less than about 10,000 mPa·s when measured at a shear rate of 100 (1/s), preferably the viscosity is less than about 6,389 mPa·s when measured at a shear rate of 100 (1/s);
having a solubility of less than about 60% when measured at pH 4, less than about 25% when measured at pH 5.5 and less than about 80% when measured at pH 7, optionally having a solubility of about 6.6 to about 20.2% when measured at pH 4, about 0.7 to about 10.1% when measured at pH 5.5 and about 7.0 to about 36.2% when measured at pH 7; optionally having a solubility of about 9.1 to about 54.6% when measured at pH 4, about 5.3 to about 20.4% when measured at pH 5.5 and about 20.3 to about 76.7% when measured at pH 7, optionally having a solubility of about 22.5 to about 54.6% when measured at pH 4, about 5.8 to about 20.4% when measured at pH 5.5 and about 20.3 to about 68.1% when measured at pH 7;
having a dry colour L* value of about 77.70 to about 97.99, a dry colour a* value of about −0.44 to about 2.03 and a dry colour b* value of about 14.34 to about 26.32, preferably wherein the dry colour L* value is about 77.70 to about 82.83, the dry colour a* value is about 0.58 to about 2.03 and the dry colour b* value is about 16.40 to about 26.32; or
a pulse protein product or a residual pulse protein product having an attribute from one or more of the following tables:
a solubility as defined in or captured by Table 3,
a dry colour as defined in or captured by Table 4;
a water binding capacity as defined or captured by Table 5;
an oil binding capacity as defined in or captured by Table 6;
a phytic acid content as defined in or captured by Table 7;
a sodium content as defined in or captured by Table 8;
an amino acid profile comprising one or more amino acids as defined or captured by Table 9;
a viscosity for a 20 wt % protein solution as defined in or captured by Table 10, and
optionally wherein the pulse protein product is derived from a pulse source selected from lentils, chickpeas, dry peas, dry beans, lupines, bambara beans, broad or fava beans, dry pigeon peas, vetches, dried cowpeas, winged beans, sword beans and yellow peas and wherein the pulse source is optionally dehulled, and
optionally wherein the food or beverage is:
i) a dairy alternative;
j) a meat alternative;
k) a seafood alternative;
l) a grain product;
m) a snack or sweet;
n) a fats and oils product;
o) a condiment or sauce; or
p) a nutritional product
q) a sports drink;
r) an energy drink; or
s) a smoothie.

94. A pet food, animal feed, industrial product, cosmetic product or personal care product comprising a pulse protein product which is defined as:
having a sodium content of less than about 0.40% d.b. or preferably less than about 0.30% d.b. more preferably less than about 0.12% d.b.;
having viscosity for a 20 wt % protein solution of less than about 10,000 mPa·s when measured at a shear rate of 100 (1/s), preferably the viscosity is less than about 6,389 mPa·s when measured at a shear rate of 100 (1/s);
having a solubility of less than about 60% when measured at pH 4, less than about 25% when measured at pH 5.5 and less than about 80% when measured at pH 7, optionally having a solubility of about 6.6 to about 20.2% when measured at pH 4, about 0.7 to about 10.1% when measured at pH 5.5 and about 7.0 to about 36.2% when measured at pH 7; optionally having a solubility of about 9.1 to about 54.6% when measured at pH 4, about 5.3 to about 20.4% when measured at pH 5.5 and about 20.3 to about 76.7% when measured at pH 7, optionally having a solubility of about 22.5 to about 54.6% when measured at pH 4, about 5.8 to about 20.4% when measured at pH 5.5 and about 20.3 to about 68.1% when measured at pH 7;
having a dry colour L* value of about 77.70 to about 97.99, a dry colour a* value of about −0.44 to about 2.03 and a dry colour b* value of about 14.34 to about 26.32, preferably wherein the dry colour L* value is about 77.70 to about 82.83, the dry colour a* value is about 0.58 to about 2.03 and the dry colour b* value is about 16.40 to about 26.32; or
a pulse protein product or a residual pulse protein product having an attribute from one or more of the following tables:
a solubility as defined in or captured by Table 3,
a dry colour as defined in or captured by Table 4;
a water binding capacity as defined or captured by Table 5;
an oil binding capacity as defined in or captured by Table 6;
a phytic acid content as defined in or captured by Table 7;
a sodium content as defined in or captured by Table 8;
an amino acid profile comprising one or more amino acids as defined or captured by Table 9;
a viscosity for a 20 wt % protein solution as defined in or captured by Table 10, and
optionally wherein the pulse protein product is derived from a pulse source selected from lentils, chickpeas, dry peas, dry beans, lupines, bambara beans, broad or fava beans, dry pigeon peas, vetches, dried cowpeas, winged beans, sword beans and yellow peas and wherein the pulse source is optionally dehulled.
